(19) United States
(12) Patent Application Publication
(10) Pub. No.: US 2024/0350561 A1
(43) Pub. Date: Oct. 24, 2024

(54) PROBIOTIC BACTERIA COMPOSITION FOR INHIBITING FUNGAL PROLIFERATION

(71) Applicant: Lactobio A/S, Copenhagen (DK)

(72) Inventors: Charlotte Vedel, Copenhagen (DK); Jens Edward Tesdorpf, Copenhagen (DK); Soren Kjaerulff, Copenhagen (DK); Helena Falholt Elvebakken, Copenhagen (DK)

(73) Assignee: Lactobio A/S, Copenhagen (DK)

(21) Appl. No.: 18/684,919

(22) PCT Filed: Aug. 25, 2022
(86) PCT No.: PCT/EP2022/073726

§ 371 Date: Feb. 20, 2024

(30) Foreign Application Priority Data
Aug. 25, 2021 (DK) PA 2021 00835
May 31, 2022 (DK) PA 2022 00503

(51) Int. Cl. A61K 35/741; A61K 31/7088; A61K 35/00; A61P 17/00; A61P 31/10

(52) U.S. Cl. CPC A61K 35/741; A61K 31/7088; A61P 17/00; A61P 31/10; A61K 2035/115

(57) ABSTRACT

The present invention relates to a composition comprising one or more isolated probiotic bacterial strains, two or more active of the one or more isolated probiotic bacterial strains, or the combination of one or more isolated probiotic bacterial strains and two or more active of the one or more isolated probiotic bacterial strains; and an acceptable carrier, wherein the one or more isolated probiotic bacterial strains and/or the two or more active of the one or more isolated probiotic bacterial strains is capable of inhibiting fungal proliferation.

### TECHNICAL FIELD OF THE INVENTION

The present invention relates to antimicrobial compositions comprising microbial-derived active materials., and more particularly the present invention relates to a composition comprising probiotic bacteria or a postbiotic fraction produced by a probiotic bacteria effective to inhibiting fungal proliferation, such as proliferation of Malassezia furfur and treat Seborrheic Dermatitis (SD).

### BACKGROUND OF THE INVENTION

Seborrheic dermatitis is a histopathological eczematous dermatosis characterized by poorly demarcated scaley erythematous patches with yellowish greasy scales. “Dandruff” is a mild form of this condition localized to the scalp. This disease may involve anyone, several, or all, of the following sites: scalp, eyebrows, glabella, paranasal and chin folds, ears and retroauricular sulci, presternal interscapular regions, pubic regions and intergluteal folds.

Corticosteroids with tar, sulfur, or antibiotics give temporary control in some cases, but often the condition is chronic and returns when the treatments are terminated. Current therapy consists of topical and systemic antimicrobials, corticosteroids and topical tar. The infecting organisms associated with these skin conditions may spread to other skin areas and may even be transmitted to other people and can result in changes of the natural skin microbiota causing a dysfunctional microbiota which further worsen the disease. Present therapy consists of topical and systemic antibiotics and antimicrobials. Traditional treatments of seborrheic dermatitis have significant impact on the scalp and especially the microbiota, therefore there is a need for a less harsh treatment and a need for reestablishment of the dysfunctional microbiota associated with seborrheic dermatitis.

Seborrheic dermatitis corresponds to excessive and visible desquamation of the scalp resulting from the excessively rapid multiplication of the epidermal cells and from their abnormal maturation. A variety of factors can induce this condition, such as excessive hair treatments, extreme climatic conditions, nervousness, diet, fatigue, or pollution. However, seborrheic dermatitis, and especially the continuously recurrence, most commonly arises from a disorder of the microbiota of the scalp and more particularly from the excessive colonization by a fungus that belongs to the family of the yeasts of the genus Malassezia, which is naturally present on the scalp.

Malassezia colonize the skin of a variety of mammals, domestic animals and birds. These lipophilic yeasts inhabit the stratum corneum of the skin of humans and animals, because this layer is rich in lipids. For reasons currently unknown, these yeasts can change their saprophytic state and invade the stratum corneum as pathogens.

A form of seborrheic dermatitis is cradle cap in infants. Infants with cradle cap have slightly red scaly or crusty yellow patches on the scalp. It may also start on the face or diaper area and spread to other part of the body.

Malassezia is also infecting the skin of animals and common in horses, cattle, sheep, canine and feline dermatitis. Malassezia is normally found on the skin, but its abnormal overgrowth can cause dermatitis, or inflammation of the skin, ears, oral cavity and body orifices.

Seborrheic dermatitis including milder versions, often referred to as dandruff, affects up to 50 percent of the world's population and affects both men and women, generally has a negative psychosocial impact. Dandruff is disagreeable aesthetically (due to the visible presence of dead skin flakes) and because of the personal discomfort felt by the individual (in particular itching). Accordingly, affected persons confronted with this problem to variable degrees wish to be rid of it efficiently and permanently.

Despite many commercially available therapies, scalp treatments and shampoos Seborrheic dermatitis remains a challenging condition, with many patients being unresponsive to several attempted therapies, making treatment unpredictable and elusive in many cases.

Many anti-dandruff treatments have been developed with the main objective of eradicating Malassezia yeasts from the scalp. Thus, the activity of the anti-dandruff active agents of today, such as zinc pyrithione, piroctone olamine, climbazole, ketoconazole, or selenium disulfide, are based mainly on their fungicidal property. Recently, other formulations using nature-based alternatives have been described, such as an anti-dandruff composition based on ellagic acid and at least one essential oil as described in WO2011/138450, or a hydrolysable tannin-enriched active substance derived from Punica granatum as described in FR2908045.

Despite the foregoing, there continues to be a need for new effective natural solutions toward inhibiting fungal proliferation and/or treating or preventing mycosis and/or treating or preventing dandruff which do not cause skin irritation and will facilitate re-establishment of a balanced natural scalp microbiota.

### SUMMARY OF THE INVENTION

The present invention is premised on the realization that various combinations of anti-dandruff actives derived from probiotic microorganisms are formulated in a composition for in particular topical use still comprising the microbial-derived active materials against Seborrheic Dermatitis (SD), and more particularly to a composition comprising probiotic bacteria or a postbiotic fraction produced by probiotic microorganisms effective to inhibit a fungi having a genus from the family of Malasseziaceae, such as Malassezia furfur, and treat Seborrheic dermatitis.

An aspect of the present invention relates to a composition comprising:
one or more isolated probiotic bacterial strains; two or more active of the one or more isolated probiotic bacterial strains; or the combination of one or more isolated probiotic bacterial strains and two or more active of the one or more isolated probiotic bacterial strains, and
an acceptable carrier,
wherein the one or more isolated probiotic bacterial strains and/or the two or more active of the one or more isolated probiotic bacterial strains is capable of inhibiting fungal proliferation.

A further aspect of the present invention relates to an anti-dandruff composition that comprises (or consists essentially of) a combination of at least two anti-dandruff actives derived from probiotic microorganisms. The anti-dandruff actives are present in the anti-dandruff composition in an effective amount to treat or prevent dandruff.

Yet an aspect of the present invention relates to the use in the prevention and/or treatment of mycoses in a human or in an animal.

The present invention also seeks to provide compounds in a hair care formulation, where the compound may provide comparable or improved, anti-fungal properties, like anti-dandruff properties compared to existing anti-dandruff agents.

An aspect of the present invention relates to a topical hair care composition comprising a combination of the composition according to the present invention and a shampoo matrix with pH below 6.5 comprising at least one detersive surfactant selected from the group consisting of an anionic surfactant, a nonionic surfactant, an amphoteric surfactant, or a combination thereof.

An aspect of the present invention relates to a leave-on composition for treatment of mycosis.

The present invention also seeks to provide the use of compounds as anti-dandruff agents, and formulations comprising said compounds for use in reducing dandruff on human skin.

In accordance with another embodiment of the present invention, an anti-dandruff hair care composition is provided that is suitable for the treatment of dandruff against Malassezia furfur.

The composition according to the present invention may include one or more isolated probiotic bacterial strains and/or two or more active of the one or more isolated probiotic bacterial strains, like anti-dandruff actives, an acceptable carrier, and has a pH below 6.5.

It was surprising to determine a synergistic effect between the anti-fungal effect, e.g. the anti-dandruff actives, and the pH of the composition. Without being bound by theory it is believed that this synergistic effect was identified as at least two different mechanisms and was observed for both viable probiotic microorganisms as well as any fraction therefrom still comprising the anti-fungal effects, in particular anti-dandruff actives. Thus, a synergy may have been observed between the antimicrobial activity of the actives against inhibition of fungi may be a genus from the family of Malasseziaceae, e.g. Malassezia furfur, as well as a synergy was observed between pH of the anti-dandruff composition and the ability of the natural microbiota to re-establish after treatment, and thereby allowing for a mild anti-fungal treatment, such as mild anti-dandruff treatment with a longer and more persistent effect as compared to traditional treatments which do not repair or facilitate repair of the dys-functional microbiota left on the scalp after anti-microbial treatments.

As a further benefit of the invention, probiotic treatment did not cause any irritation of the human or animal body or on the skin or scalp.

In accordance with yet another embodiment of the present invention, a method for treating dandruff conditions associated with the proliferation of yeasts of the Malassezia genus on a scalp of a subject is provided. The method comprises applying an effective amount of the anti-dandruff composition to the scalp of the subject, wherein the effective amount of the anti-dandruff composition inhibits the proliferation of the yeasts of the Malassezia genus on the scalp.

The present invention will now be described in more detail in the following.

### DETAILED DESCRIPTION OF THE INVENTION

The present invention may relate to a composition, a use of said composition and a method for use to inhibit fungal proliferation, prevent or treat scalp conditions associated with a dysfunctional microbiota. The composition, e.g. an anti-dandruff composition, may comprise functional probiotic bacteria and/or metabolites obtained by fermentation of probiotic bacteria.

Anti-dandruff compositions, particularly shampoos, are well known and have been commercially available for many years. Many anti-dandruff actives have been used commercially such as ketoconazole, zinc pyrithione, piroctone olamine, octopirox, salicylic acid, selenium sulphide, coal tar, and azelaic acid.

These type of actives, may preferably be active compounds generally function as anti-microbial/fungal agents, being effective against certain species and strains of fungi, yeast and/or bacteria, in particular being effective against fungi and/or yeast. For example, the yeast-like fungus Malassezia lives on the scalp of most adults, but for some people it irritates the scalp and can cause more skin cells to grow. Although Malassezia yeasts are a part of the normal microflora, under certain conditions they can cause superficial skin infection. These extra skin cells die and fall off, making them appear white and flaky in hair and on clothes. Thus, materials which are active against Malassezia, in particular the species Malassezia furfur, can reduce the severity of dandruff.

Preferably, the one or more active of the one or more probiotic bacterial strains (preferably isolated probiotic bacterial strains) may be provided in an effective amount to inhibit fungal proliferation.

These shampoos and orther topic antifungal preparations are often combined with a cortisonic drug to control the inflammation and alleviate the pain and itching. However, the use of these molecules may not produce satisfactory results, and in some cases these compounds exhibit an intrinsic and undesired cytotoxicity as well as damage to the microbiota.

There is a continual requirement for improved anti-dandruff actives and end-use products containing anti-dandruff actives non-irritant to the skin and that do not have the damaging effect on the microbiota of the scalp.

The present invention may provide an effective inhibition, treatment or prevention when applied topically, which do not have the damaging effect on the natural microbiota.

A preferred embodiment of the present invention relates to a composition comprising:
one or more isolated probiotic bacterial strains; two or more active of the one or more isolated probiotic bacterial strains; or the combination of one or more isolated probiotic bacterial strains and two or more active of the one or more isolated probiotic bacterial strains, and
an acceptable carrier,
wherein the one or more isolated probiotic bacterial strains and/or the two or more active of the one or more isolated probiotic bacterial strains is capable of inhibiting fungal proliferation.

Preferably the fungi capable of fungal proliferation may be a fungi of the genus selected from the family of Malasseziaceae. Preferably the genus from the family of Malasseziaceae may be Malassezia.

A further preferred embodiment of the present invention relates to a composition (e.g. an anti-dandruff composition) comprising an effective amount of a probiotic microorganism able to produce actives (e.g. anti-dandruff actives) or a fraction of the probiotic microorganisms wherein the fraction comprises the actives.

According to another aspect of the present invention there is provided a method of providing anti-fungal efficacy, in particular anti-dandruff efficacy, which comprises the steps of:
(i) wetting the hair with water;
(ii) applying an effective amount of a composition according to the present invention comprising a probiotic microorganism or a fraction thereof
(iii) rinsing the composition from the hair using water; and
(iv) optionally repeating steps (ii) and (iii).

According to another aspect of the present invention there is provided a method of providing anti-fungal efficacy, which comprises the steps of applying a leave-on composition to skin, epithelium, nails, mouth, including the skin of the scalp, feet, vagina, genitalia and ears.

According to another aspect of the present invention there is provided a method for killing or retarding the growth of a fungus, in particular a fungus from the family of Malasseziaceae, such as Malassezia spp., the method comprising the step of contacting the fungus, such as Malassezia spp. with a composition comprising actives derived from a probiotic microorganism according to the present invention.

As used herein, the terms “for example”, “for instance”, “such as”, or “including” are meant to introduce examples that further clarify more general subject matter. Unless otherwise specified, these examples are provided only as an aid for understanding the applications illustrated in the present disclosure, and are not meant to be limiting in any fashion.

The term “anti-fungal composition” relates to a composition according to the present invention, capable of inhibiting fungal proliferation, treat or prevent fungal infection, e.g. treat or prevent mycoses and/or dandruff in a human or animal.

Even the anti-dandruff effect is the effect primarily described in the present invention, the composition according to the present invention also show strong anti-mycosis effects.

The fungal proliferation may result in development of dandruff and/or mycosis in a mammal. By limiting, reducing or terminating the fungal proliferation it is believed that development of dandruff and/or mycosis may be preventer, avoided or limited.

Preferably, the composition according to the present invention may be an anti-dandruff composition and or an anti-mycosis composition.

The term “anti-dandruff composition” refers to the provision of effects for preventing and/or treating scalp dandruff. This includes preventing and/or reducing excessive dandruff formation, and/or visually unappealing excessively formed dandruff.

The term “actives” may relate to compounds active against fungal proliferation, for the treatment and/or prevention of mycoses in a human or animal. The actives may preferably include a bacteriocin, an organic acid, a cell wall material, or a combination hereof.

The present invention relates to an anti-fungal composition, in particular an anti-dandruff composition, and methods of using the composition to treat or inhibit anti-fungal proliferation, such as for the treatment or inhibition of dandruff. The composition according to the present invention may be useful for application to keratinous tissue or scalp surface and comprise an effective amount of a combination of actives, which inhibit, reduce, or eliminate symptoms, e.g. dandruff symptoms, arising from the proliferation of Malassezia furfur. The compositions of the present invention may be in a wide variety of product forms that include, but are not limited to, solutions, suspensions, lotions, creams, gels, ointments, oils, emulsions, sprays, aerosols, shampoos, hair conditioners, pastes, foams, powders, mousses, wipes, strips, patches, hydrogels, film-forming products, and the like. The compositional form may follow from the particular dermatologically-acceptable carrier chosen.

In an embodiment of the present invention the anti-dandruff composition may include compositions that are applied to the hair and/or the skin underneath the hair and the anti-dandruff composition may comprise (or consist essentially of) at least one of various combinations of probiotic derived actives and a dermatologically-acceptable carrier, the combinations of which are effective to inhibit Malassezia furfur and treat dandruff.

The active may refers to a single compound or a composition comprising two or more compounds that possesses an ability to inhibit the proliferation of fungi, such as Malassezia furfur, when present in a composition in an effective amount.

The term “effective amount,” as used herein, means an amount of a compound or composition comprising two to more compounds, is sufficient to reduce or inhibit the proliferation of fungi, in particular Malassezia furfur, reduce or inhibit the visible and/or physical effects of fungal infection, mycosis or dandruff caused by fungal proliferation, such as the proliferation of Malassezia furfur, or reduce or inhibit scalp pruritus, by a statistically significant amount.

The term “topical application” as used herein, means to apply or spread the compositions of the present invention onto the surface of the hair, skin eg. the scalp.

The term “acceptable carrier” as used herein, means a carrier suitable internal (e.g. orally or rectally intake) or external body (topical) use without undue toxicity, incompatibility, instability, allergic response, discomfort and the like.

The term “dermatologically-acceptable,” as used herein, means that the compositions or components thereof so described are suitable for use in contact with mammalian keratinous or skin tissue without undue toxicity, incompatibility, instability, allergic response, and the like.

In accordance with embodiments of the present invention, the composition, e.g. the anti-dandruff composition comprises (or consists essentially of) an effective amount of a combination of at least two actives selected from the group organic acids. In a further preferred embodiment the organic acids are selected from the group consisting of lactic acid, citric acid, acetic acid, malic acid, tartaric acid, 3-phenyllactic acid, 3-hydroxy phenyllactic acid, 4-hydroxy phenylactic acid, propionic acid and succinic acid, salicylic acid, azelaic acid, indole-3-lactic acid, indole-3-acetic acid, 2-hydroxybuturic acid, 2-Hydroxyisocaproic acid, 3-(R)-hydroxydecanoic acid, 3-hydroxy-5-cic dodecanoic acid, 3-(R)-hydroxy dodecanoic acid, 3-(R)-hydroxy tetradecanoic acid, glycolic acid, and N-acetylaspartic acid.

In an embodiment of the present invention the composition may comprise a viable probiotic microorganism. In a further embodiment, of the present invention the active present in the composition may be in the form of a viable probiotic microorganism. Preferably, the viable probiotic microorganism may be able to inhibit fungal proliferation, e.g. inhibit Malassezia furfur. Preferably, the inhibition of Malassezia furfur may be obtained when the composition may be applied in a topical application to the scalp or dandruff affected area of the skin.

The term “viable” or “live” as used herein relates to a microorganism which is not dead and are able to have an active metabolism.

The term “microbiota” as used herein relates to communities of commensal, symbiotic and pathogenic microorganisms found in and on all multicellular organisms. Microbiota include bacteria, archaea, protists, fungi, yeast, viruses and phages.

The term “microbiota dysfunction” as used herein relates to a state in which the microbiota functions incorrectly or is obstructed from functioning at all. Unless otherwise specified, a microbiota dysfunction is in view of the present invention including the overgrowth or increase in growth of a pathogenic microorganism resulting in a dysfunctional microbiota. One example of a dysfunctional microbiota is an increase in Malassezia furfur resulting in mycosis, or Seborrhoeic dermatitis, like dandruff.

In an embodiment of the present invention fungal infection may be mycosis or Seborrhoeic dermatitis. Preferably, seborrhoeic dermatitis may be dandruff.

The term “Probiotic microorganism” as used herein relates to live microorganisms that are intended to have health benefits when consumed or applied to the host. Examples of suitable probiotic microorganisms include yeasts such as Saccharomyces, Debaromyces, Candida, Pichia and Torulopsis, moulds such as Aspergillus, Rhizopus, Mucor, and Penicillium and Torulopsis and bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Cutibacterium, Lacticaseibacillus, Levilactobacillus, Lactiplantibacillus and Lactobacillus.

The most commonly used probiotics are strains of the lactic acid bacteria (LAB). The term “lactic acid bacteria” includes species from the families Lactobacillus, Aerococcaceae, Bifidobacteriaceae, Carnobacteriaceae, Enterococcaceae, Leuconostocaceae and Streptococcaceae. These are considered non-pathogenic and are used as probiotic bacteria in general to improve gastrointestinal flora and in the treatment of gastrointestinal symptoms. The present invention relates to topical application of probiotics characterized by the probiotic producing anti-dandruff actives.

The microorganism may preferably be lactic acid bacteria. The microorganism may even more preferably be selected among the genera Lactobacillus, Lactiplantbacillus, Holzapfelia, Amylolactobacillus, Bombilactobacillus, Companilactobacillus, Lapidilactobacillus, Agrilactobacillus, Schleiferilactobacillus, Loigolactobacillus, Lacticaseibacillus, Latilactobacillus, Dellaglioa, Liquorilactobacillus, Ligilactobacillus, Furfurilactobacillus, Paucilactobacillus, Limosilactobacillus, Fructilactobacillus, Acetilactobacillus, Apilactobacillus, Levilactobacillus, Secundilactobacillus, Lentilactobacillus, Leuconostoc, Bifidobacterium, Pediococcus, Lactococcus, Streptococcus, Aerococcus, Carnobacterium, Enterococcus, Oenococcus, Sporolactobacillus, Tetragenococcus, Vagococcus, and Weissella.

The preferred microorganisms may in particular be bacteria. The probiotic bacteria may preferably be selected from the group comprising Lactococcus lactis, Lacticaseibacillus rhamnosus, Lactiplantibacillus plantarum, Lactobacillus helveticus, Lactobacillus jensenii, Lactobacillus acidophilus, Lactobacillus bulgaricus, Lactobacillus amylovorus, Lactobacillus amylolyticus, Lactobacillus alimentarius, Lactobacillus aviaries, Lactobacillus delbrueckii, Lactobacillus diolivorans, Lactobacillus farciminis, Lactobacillus gallinarum, Lacticaseibacillus casei, Lactobacillus crispatus, Lactobacillus gasseri, Lactobacillus johnsonii, Lactobacillus hilgardii, Lactobacillus kefiranofaciens, Lactobacillus kefiri, Lactobacillus mucosae, Lactobacillus panis, Lactiplantibacillus paraplantarum, Lactobacillus pontis, Latilactobacillus sakei, Lactobacillus saliverius, Lactobacillus sanfraciscensis, Lacticaseibacillus paracasei, Lactobacillus pentosus, Lactobacillus cellobiosus, Lactobacillus collinoides, Lactobacillus coryniformis, Lactobacillus curvatus, Levilactobacillus brevis, Lactobacillus buchneri, Lactobacillus fructivorans, Lactobacillus hilgardii, Lactobacillus fermentum, Lactobacillus reuteri, Lactobacillus ingluviei, Weissella viridescens, Bifidobacterium bifidum, Bifidobacterium adolescentis, Bifidobacterium breve, Bifidobacterium longum, Bifidobacterium animalis, Carnobacterium divergens, Corynebacterium glutamicum, Leuconostoc citreum, Leuconostoc lactis, Leuconostoc mesenteroides, Leuconostoc pseudomesenteroides, Oenococcus oeni, Pasteuria nishizawae, Pediococcus acidilactici, Pediococcus dextrinicus, Pediococcus parvulus, Pediococcus pentosaceus, Probionibacterium freudenreichii, Probionibacterium acidipropoinici, Enterococcus faecium, Enterococcus faecalis, Streptococcus thermophilus, Bacillus amyloliquefaciens, Bacillus atrophaeus, Bacillus clausii, Bacillus coagulans, Bacillus flexus, Bacillus fusiformis, Bacillus lentus, Bacillus licheniformis, Bacillus mega-terium, Bacillus mojavensis, Bacillus pumilus, Bacillus smithii, Bacillus subtilis, Bacillus vallismortis, Geobacillus stearother-mophilus or mutants thereof.

In one preferred embodiment of the invention the composition comprises at least one strain selected from the group consisting of Lactiplantibacillus plantarum LB356R (DSM 33094), Lactiplantibacillus plantarum LB244R (DSM 32996), Weissella viridescens LB10G (DSM 32906), Lacticaseibacillus paracasei LB113R (DSM 32907), Lacticaseibacillus paracasei LB116R (DSM 32908), Levilactobacillus brevis LB152G (DSM 32995), Lacticaseibacillus paracasei LB28R (DSM 32994), Enterococcus faecium LB276R (DSM 32997), Leuconostoc mesenteriodes LB349R (DSM 33093), Lactiplantibacillus plantarum LB316R (DSM 33091), Lactiplantibacillus plantarum LB312R (DSM 33098), Pediococcus pentosaceus LB606R (DSM 33730), Lactiplantibacillus plantarum LB679R (DSM 33731), Lactobacillus crispatus LB714R (DSM 33732) Lactobacillus gasseri LB905R (DSM 34094), Lactobacillus crispatus LB912R (DSM 34095), Lactobacillus jensenii LB918R (DSM 34096), Lactobacillus crispatus LB919R (DSM 34097), Lacticaseibacillus paracasei subsp. paracasei LB555R (DSM 34249), Lactiplantibacillus plantarum LB681R (DSM 34250); and/or any combinations hereof or mutant strains thereof and/or the cell lysate and/or the soluble metabolite of anyone of these probiotic strains.

The number of microorganisms is measured as Colony Forming Units (CFU) per ml or per gram.

The microorganisms according to the present invention may preferably be in isolated or purified form, where the term “isolated” means in particular that the lactic acid bacteria are derived from their culture medium including their natural medium, for example. The terms “isolated” or “purified” may not be restricted to absolute purity. The terms “isolated” and “purified” may be used interchangeable.

In an embodiment of the present invention the probiotic strain may be used as a live isolated microorganism in a stabilized form. Suitable methods for stabilization are known to those skilled in the art and includes freeze drying or lyophilization involving different cryoprotectants.

In a further embodiment of the present invention the strain may be used as a live isolated strain.

Preferably, the strain may be used as a live isolated strain. Preferably, the strain may be used as a live isolated stabilized strain. Even more preferably, the strain may be used as a live isolated strain stabilized by lyophilization. Even more preferably, the strain may be used as a live isolated strain stabilized by lyophilization and comprising a cryoprotectant.

In a preferred embodiment of the invention the probiotic strain is a Gram-positive bacteria.

In one preferred embodiment of the invention the composition comprises at least one strain selected from the group consisting of Lactiplantibacillus plantarum LB356R (DSM 33094), Lactiplantibacillus plantarum LB244R (DSM 32996), Weissella viridescens LB10G (DSM 32906), Lacticaseibacillus paracasei LB113R (DSM 32907), Lacticaseibacillus paracasei LB116R (DSM 32908), Levilactobacillus brevis LB152G (DSM 32995), Lacticaseibacillus paracasei LB28R (DSM 32994), Enterococcus faecium LB276R (DSM 32997), Leuconostoc mesenteriodes LB349R (DSM 33093), Lactiplantibacillus plantarum LB316R (DSM 33091), Lactiplantibacillus plantarum LB312R (DSM 33098), Pediococcus pentosaceus LB606R (DSM 33730), Lactiplantibacillus plantarum LB679R (DSM 33731), Lactobacillus crispatus LB714R (DSM 33732), Lactobacillus gasseri LB905R (DSM 34094), Lactobacillus crispatus LB912R (DSM 34095), Lactobacillus jensenii LB918R (DSM 34096), Lactobacillus crispatus LB919R (DSM 34097), Lacticaseibacillus paracasei subsp. paracasei LB555R (DSM 34249), Lactiplantibacillus plantarum LB681R (DSM 34250) or mutant strains hereof.

The term “postbiotic” refers to compounds, metabolites or cell materials secreted or released from a probiotic microorganism providing a health benefit when applied to the host. A postbiotic composition is characterized by having a health benefit without the presence of the viable microorganism.

The term “Postbiotic fraction of a probiotic microorganism” as used herein disclose a fermented composition of the probiotic microorganism substantially free from viable microorganisms. The composition can comprise cell material including dead cells.

In an embodiment of the present invention the composition may have a pH value below pH 8.0, such as below pH 7.5, e.g. below pH 7.0, such as below pH 6.5, e.g. below pH 6.0, such as below pH 5.5, e.g. below pH 5.0, such as below pH 4.75, e.g. below pH 4.5, such as below pH 4.25, e.g. below pH 4.0, such as below pH 3.75, e.g. below pH 3.5, such as below pH 3.25.

The preferred pH of the composition will be pH 2.5 to pH 7, more preferred from pH 3 to pH 6.5, and even more preferred from pH 3.5 to pH 5.5. The low pH of the composition resulting from the acids produced by the probiotic microorganism will when applied on scalp cause acidification of the surface of skin with elevated pH. Healthy scalp has a pH at about 4.5 and the metabolites produced by the probiotic microorganism will as another beneficial effects assist in maintaining a healthy pH of the skin.

In an embodiment of the present invention the at least two actives may be produced by metabolism of the isolated viable probiotic bacterial strain.

In an further embodiment of the present invention the least two actives may be produced by a single isolated probiotic bacterial strain.

Preferably, the actives may be selected from a bacteriocins, an organic acid, a cell wall material, or a combination hereof.

In one preferred embodiment of the present invention the active ingredient being an organic acid is present in the protonated form of the acid and PH is equal to the pKa of the acid or below.

This invention is based upon the discovery that some species of lactic acid bacteria will produce bacteriocin in the supernatant in an amount effective to inhibit growth of Malassezia even though the lactic acid bacteria are no longer present.

In one embodiment of the invention the preferred microorganism is an isolated wild type lactic acid bacteria.

In one embodiment the preferred bacteriocin used in the present invention is produced by a probiotic bacteria, in a further preferred embodiment the bacteriocin is produced by a lactic acid bacteria, in a further preferred embodiment the bacteriocin is produced by one of the following bacteria; Lactiplantibacillus plantarum LB356R (DSM 33094), Lactiplantibacillus plantarum LB244R (DSM 32996), Weissella viridescens LB10G (DSM 32906), Lacticaseibacillus paracasei LB113R (DSM 32907), Lacticaseibacillus paracasei LB116R (DSM 32908), Levilactobacillus brevis LB152G (DSM 32995), Lacticaseibacillus paracasei LB28R (DSM 32994), Enterococcus faecium LB276R (DSM 32997), Leuconostoc mesenteriodes LB349R (DSM 33093), Lactiplantibacillus plantarum LB316R (DSM 33091), Lactiplantibacillus plantarum LB312R (DSM 33098), Pediococcus pentosaceus LB606R (DSM 33730), Lactiplantibacillus plantarum LB679R (DSM 33731), Lactobacillus crispatus LB714R (DSM 33732), Lactobacillus gasseri LB905R (DSM 34094), Lactobacillus crispatus LB912R (DSM 34095), Lactobacillus jensenii LB918R (DSM 34096), Lactobacillus crispatus LB919R (DSM 34097), Lacticaseibacillus paracasei subsp. paracasei LB555R (DSM 34249), Lactiplantibacillus plantarum LB681R (DSM 34250); and/or any combinations hereof.

The bacteriocin is preferably used in the composition of the present invention in an amount between 1 and 1,000,000 Arbitrary Units (AU) of bacteriocin. Once AU of bacteriocin was defined as 5 microliters of the highest dilution of culture supernatant yielding a definite zone of growth inhibition with a lawn of an indicator strain on an agar plate.

In an embodiment of the invention the active may be a bacteriocin.

The term “bacteriocin” refers to an antimicrobial peptide or protein produced by a bacteria that is active against microorganisms but does not harm the producing bacteria. For purposes of the present invention, bacterocins or bacterocin sources generally include antimicrobial agents suitable for use in formulations as cosmetics or pharmaceuticals. Especially preferred antimicrobial agents include “lantibiotics” (i.e., polypeptides containing lanthionine and beta-methyl lanthionine). Non-limiting examples of such lantibiotics are nisin, such as nisin A or nisin Z, or nisin analogs or related lanthionine-containing peptides, such as pediocin, lactosin, lactacins (e.g., lacticin A, lacticin B, lactacin F), camocin, enterocin, plantaricin, subtilin, epidermin, cinnamycin, duramycin, ancovenin, Pep 5, and the like, individually or in any combination thereof. Other bacterocins that are useful in the present invention include, for example, lactococcins (e.g., lactococcin A, lactococcin B, lactococcin M), leucocoin, helvetican, acidophilucin, caseicin, salivarcin X, lacticin J46, lacticin 481, lacticin 3147, salivarcin A, salivarcin A2, salivarcin A3, salivarcin A4, BHT-Aa, BHT Ab, salivarcin A5, salivarcin B, streptin, salivaricin Al, streptin, streptococcin A-FF22, mutacin BNY266, mutacin 1140, mutacin K8, mutacin II, smbAB, bovicin HJ50, bovicin HC5, macedocin, leucocin C, sakacin 5X, enterocin CRL35/mundticin, avicin A, mundticin I, enterocin HF, bavaricin A, ubericin A, leucocin A, mesentericin Y105, sakacin G, curvacin A/sakacin A, lactocin 5, cyctolysin, enterocin A, divercin V41, divercin M35, bavaricin, coagulin, pediocin PA-1, mundticin, piscicocin CS526, piscicocin 126/Vla, sakacin, Pcarnobacteriocin BM1, enterocin P, piscicoin Vlb, penocin A, bacteriocin 31, bacteriocin RC714, hiracin JM79, bacteriocin T8, enterocin SE-K4, carnobacteriocin B2 and Plantaricins.

The term “plantaricin” refers to bacteriocins from Lactiplantibacillus plantarum, the major types of plantaricins includes Plantaricin A, Plantaricin E, Plantaricin F, Plantaricin J, Plantaricin K, Plantaricin C, Plantaricin D, Plantaricin W, Plantaricin T and Plantaricin S. As well as other plantaricins e.g. Plantaricin35d, Plantaricin MG, Plantaricin 423, Plantaricin 154, Plantaricin 149, Plantaricin 163, Plantaricin LC74, Plantaricin K25, Plantaricin ST31, Plantaricin SA6. In particular broad spectrum Plantaricins e.g. Plantaricin F, Plantaricin DL3, Plantaricin ZJ008, Plantaricin MG, Plantaricin Q7, Plantaricin KL-1Y, Plantaricin 163, Plantaricin 154.

Thus, an embodiment of the present invention relates to a composition comprising a probiotic microorganism producing at least one active wherein the active comprises (consist essentially of) a bacteriocin.

In yet another embodiment of the present invention at least 2 different bacteriocins may be produced by the probiotic microorganism.

The composition of the inventions comprises a probiotic microorganism being able to produce actives, such as anti-dandruff actives, or a fraction of postbiotics from the probiotic organism wherein the actives are maintained in the postbiotic composition.

In an embodiment of the present invention the actives may include an organic acid, or a combination of one or more organic acids and one or more baceriocins.

The organic acids are preferable used in the Postbiotic composition in a total concentration by weight from about 0.1 to 20%. E.g. by weight from, from 1 to 5% lactic acid or acetic acid. The organic acids are preferable selected from lactic acid, citric acid, acetic acid, malic acid, tartaric acid, phenyllactic acid, 3-hydroxy phenyllactic acid, 4-hydroxy phenylactic acid, propionic acid and succinic acid, salicylic acid, azelaic acid, indole-3-lactic acid, indole-3-acetic acid, 2-hydroxybuturic acid, 2-Hydroxyisocaproic acid, 3-(R)-hydroxydecanoic acid, 3-hydroxy-5-cic dodecanoic acid, 3-(R)-hydroxy dodecanoic acid, 3-(R)-hydroxy tetradecanoic acid, glycolic acid, and N-acetylaspartic acid is preferable used in the concentrations from 0.1 to 10% (w/w) in the postbiotic composition.

As noted above, the active portion of the composition comprises (or consists essentially of) a combination of at least two actives derived from a probiotic microorganism or applied in form of a viable probiotic microorganism being able to produce at least two actives.

In an embodiment of the present invention the active may be produced by a probiotic bacterium and the active may be selected from; lactic acid, citric acid, acetic acid, malic acid, tartaric acid, 3-phenyllactic acid, 3-hydroxy phenyllactic acid, 4-hydroxy phenylactic acid, propionic acid and succinic acid, salicylic acid, azelaic acid, indole-3-lactic acid, indole-3-acetic acid, 2-hydroxybuturic acid, 2-Hydroxyisocaproic acid, 3-(R)-hydroxydecanoic acid, 3-hydroxy-5-cic dodecanoic acid, 3-(R)-hydroxy dodecanoic acid, 3-(R)-hydroxy tetradecanoic acid, glycolic acid, and N-acetylaspartic acid.

The active may be produced on the scalp by a probiotic bacterium and may be selected from; 2-Hydroxyisocaproic acid, phenyl lactic acid, salicylic acid, acetyl salicylic acid, indole-3-lactic acid, gallic acid, azelaic acid, 2-hydroxybuturic acid, N-acetylaspartic acid, succinic acid and lactic acid.

In an embodiment of the present invention the composition may comprise at least one of the following combinations of actives: bacteriocin and/or phenyl lactic acid and/or salicylic acid and/or lactic acid and/or 2-Hydroxyisocaproic acid and/or azelaic acid and/or succinic acid and/or indole-3-lactic acid and/or 2-hydroxybuturic acid and/or N-acetylaspartic acid and/or indole-3-acetic acid and/or gallic acid.

In another embodiment of the present invention the actives may include the combination of at least 2 different bacteriocins produced by a probiotic microorganism or isolated in a fraction from fermentation of a probiotic microorganism, wherein the probiotic microorganism is not genetically modified to produce bacteriocins.

The present invention may be suitable for directly use as a scalp care product or formulated into a scalp care product, in therapeutic or scalp care compositions for prevention or treatment of scalp conditions or for modulation of dysfunctional microbiotas.

The probiotic microorganisms of the invention may be able to produce a yield of functional metabolites e.g. actives, like the anti-dandruff actives, during growth sufficient to provide a broad spectrum antimicrobial activity, anti-inflammatory activity, peeling effects, moisturizing effects and/or functional effects, such as on feet, nails, and scalp firmness. Without being bound by theory, it is believed that the effects may be provide by activation of fibrillin and collagen synthesis.

Further it was surprising to determine a synergistic effect between the actives or metabolites present in the composition, and the pH of the composition. This synergistic effect may allow the functional concentration of each active to be lower than the functional concentration needed with a purified active.

E.g. azelaic acid may be used in high concentrations. In an embodiment of the present invention the concentration of azelaic acid may be between 2-20% (w/w), using azelaic acid in a composition comprising multiple actives or metabolites all contributing to a synergistic functional effect and thereby the concentration in use can be significantly reduced, also reducing side effects and any toxicity or irritation which can be observed while using these compounds in the high concentrations >2% (w/w).

The present invention relates to a composition comprising an active produced by growth of a probiotic microorganism. The composition may be administered for internal use or external use. The external use may be topical application, e.g. to the scalp, to the feet, and/or to the nails as a viable microorganism or applied as a postbiotic composition comprising the actives.

The present invention may relate to a composition comprising anti-dandruff active produced by growth of a probiotic microorganism and applied topical to the scalp as a viable microorganism or applied as a postbiotic composition comprising the anti-dandruff actives.

In an embodiment of the present invention the composition comprising a probiotic microorganism able to produce a bacteriocin and at least one organic acid as actives.

In another embodiment of the invention the composition comprising a probiotic microorganism able to produce a plantaricin and at least one organic acid as actives.

Preferably, the composition comprise at least 2 different bacteriocins as actives.

The composition of the present invention may comprise a probiotic microorganism being able to produce actives.

The composition of the present invention may comprise an acceptable carrier, for the topical composition the acceptable carrier may be a dermatologically-acceptable carrier (which may be referred to as “carrier”) for the providing the actives. A suitable carrier may be selected to yield a desired product form. Furthermore, the solubility or dispersibility of the components may dictate the form and character of the carrier. In one embodiment, the carrier may be present at a level of from about 30 wt percent to about 99 wt percent, about 40 wt percent to about 98 wt percent, about 50 wt percent to about 96 wt percent, or, alternatively, from about 60 wt percent to about 95 wt percent, by weight of the composition. Wt percent is based on the weight of the entire composition.

The carrier may be in a wide variety of forms. Non-limiting examples include simple solutions (e.g., aqueous, organic solvent, or oil based), emulsions, and solid forms (e.g., gels, powders, sticks, flowable solids, or amorphous materials). In certain embodiments, the carrier is an aqueous carrier, which may comprise water or natural botanical juices, such as aloe vera water.

In certain embodiments, the carrier may be in the form of an emulsion. Emulsion may be generally classified as having a continuous aqueous phase (e.g., oil-in-water and water-in-oil-in-water) or a continuous oil phase (e.g., water-in-oil and oil-in-water-in-oil). The oil phase of the present invention may comprise natural oils, vegetable oils, silicone oils, nonsilicone oils such as hydrocarbon oils, esters, ethers, and the like, and mixtures thereof.

For emulsions, the aqueous phase may comprise water, such as demineralized or distilled water, For example. Other acceptable carriers that may be used in the aqueous carrier include, but are not limited to alcohol or ether compounds, such as ethanol, glycerol, dipropylene glycol, propylene glycol, butylene glycol, 1,4-butanediol, 3-allyloxy-I, 2-propanediol, dipropylene glycol n-butyl ether, 1,2-hexanediol, dimethyl isosorbide, ethanol, I,3-butanediol, 1,3-propanediol, 2,2′-thiodiethanol, and 1,6-hexanediol, or combinations thereof.

In one embodiment of the invention the carrier and the active may be separated into two compartments, followed by mixing the content of the two compartments into one composition just before administration, such as administration by topical application.

For example, a bottle comprising the carrier and a cap with a compartment comprising a lyophilized viable probiotic microorganism which may be released from the cap-compartment into the carrier before administration.

The compositions according to the present invention may have a pH ranging from about 3.0 to about 6.5, which may be measured by taking a direct pH measurement using a standard hydrogen electrode of the composition at 25 degrees centigrade Accordingly, the pH of the composition may be within the range from about 3 to about 6, more preferable within the range from 3.5 to 5.5.

A preferred embodiment of the invention is a composition, such as an anti-dandruff composition, with a pH below 6.5, more preferred a composition with a pH below 6, more preferred composition with a pH below 5.5 and more preferred a composition with a pH below 5.2.

In an embodiment of the present invention the composition may be formulated into a soap, a shampoo, an emulsion; an oil; a paste; a powder; a talc; a lotion; a foam; a gel; an ointment; a suspension; a mist; or a liquid; or a tablet.

In a further embodiment of the present invention, the compositions of the present invention may be prepared in typical formulations for topical application. They may preferably be in the form of solutions, dispersion, emulsions, powders, talcs, encapsulated, spheres, spongers, solid dosage forms, foams, and other delivery mechanisms.

The compositions of the embodiments of the present invention may be hair tonics, leave-on hair products such as scalp serum, conditioners, treatment, and styling products, rinse-off hair products such as conditioners, shampoos, and treatment products; and any other form that may be applied to the scalp or skin.

The composition according to the present invention may be a leave-on composition or a rinse-off composition according to the desired use.

In one preferred embodiment of the invention the composition may be an anti-dandruff composition, and preferably a leave-on scalp treatment.

In another preferred embodiment of the invention the composition may be a shampoo.

In a further embodiment of the present invention the composition may be a nail composition. In addition to the above description of the formulation of the composition, the nail composition may be a nail polish, a nail crème, a nail oil, a nail emulsion, a nail gel, an ointment or the like.

The nail composition may be suitable for removing, limiting, treating or preventing fungal infections, like mycosis or nail mycosis, on the nails of a human or an animal.

In yet an embodiment of the present invention, the composition may be a foot composition. In addition to the above description of the formulation of the composition, the foot composition may preferably be a foot crème, a foot oil, a foot emulsion, foot ointment or the like.

The foot composition may be suitable for removing, limiting, treating or preventing fungal infections, like mycosis or foot mycosis, athletes foot on the feet of a human or an animal.

### Other Ingredients of the Composition

Accordingly, the composition according to the present invention may also include other common hair ingredients. The CTFA Cosmetic Ingredient Handbook, Tenth Edition (published by the Cosmetic, Toiletry, and Fragrance Association, Inc. (now called The Personal Care Products Council), Washington, D.C.) (2004), describes a wide variety of nonlimiting materials that can be added to the composition herein. Examples of these ingredient classes include, but are not limited to: abrasives, absorbents, fragrances, pigments, colorings/colorants, essential oils, skin sensates, astringents, anti-acne agents, anti-caking agents, antifoaming agents, antimicrobial agents, antioxidants, binders, biological additives, buffering agents, bulking agents, chelating agents, film formers, opacifying agents, pH adjusters, propellants, reducing agents, sequestrants, rheology modifiers, conditioning agents, emulsifiers, and surfactants In accordance with an embodiment, the anti-dandruff composition may be formulated as a hair care composition, such as a shampoo, a hair conditioner, or a shampoo-conditioner combination, which further include one or more of the following ingredients; (i) surfactants (anionic, amphoteric/zwitterionic, non-ionic), (ii) conditioning agents, (iii) emulsifiers, (iv) opacifiers, (v) thickeners, and (vi) buffers.

### Surfactants

Accordingly, in one embodiment, the composition of the invention may be formulated as a hair care composition, with a shampoo matrix comprising at least one detersive surfactant selected from the group consisting of an anionic surfactant, a nonionic surfactant, an amphoteric surfactant, or a combination thereof.

The hair care composition of the present invention may include a detersive surfactant, which provides cleaning performance to the composition.

The concentration of the detersive surfactant component in the composition, such as in the hair care composition, should be sufficient to provide the desired cleaning and lather performance, and generally ranges from about 2 wt percent to about 50 wt percent, from about 5 wt percent to about 40 wt percent, from about 8 wt percent to about 35 wt percent, or from about 10 wt percent to about 30 wt percent. Accordingly, composition, such as the hair care composition, may comprise a detersive surfactant in an amount of about 5 wt percent, about 10 wt percent, about 12 wt percent, about 15 wt percent, about 17 wt percent, about 18 wt percent, about 20 wt percent, about 25 wt percent, about 30 wt percent, or in a range between any two of the foregoing, for example. Wt percent is based on the weight of the entire composition.

Exemplary anionic surfactants for use in the composition may include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In a further embodiment of the present invention, the anionic surfactant is sodium lauryl sulfate, sodium laureth sulfate, or a combination thereof.

Suitable amphoteric/zwitterionic surfactants for use in the composition herein may include those which are known for use in e.g. hair care or other personal care cleansing. Concentrations of such surfactants may range from about 0.5 wt percent to about 20 wt percent, and from about 1 wt percent to about 10 wt percent. Example is betaine and further nonlimiting examples of suitable zwitterionic or amphoteric surfactants are described in U.S. Pat. Nos. 5,104,646 and 5,106,609, which are incorporated herein by reference in their entirety.

Amphoteric detersive surfactants suitable for use in the composition may include those surfactants broadly described as derivatives of aliphatic secondary and tertiary amines in which the aliphatic radical can be straight or branched chain and wherein one of the aliphatic substituents contains from about 8 to about 18 carbon atoms, and contains at least one anionic group such as carboxy, sulfonate, sulfate, phosphate, or phosphonate. Zwitterionic detersive surfactants suitable for use in the hair care composition include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Exemplary amphoteric and/or zwitterionic detersive surfactants for use in the present composition include cocoamphoacetate, cocoamphodiacetate, lauroamphoacetate, lauroamphodi acetate, cocamidopropyl betaine, cocamidopropyl hydroxysultaine, and mixtures thereof.

Nonionic Surfactants may be added to some compositions, in particular to shampoo compositions.

The composition according to the present invention, such as the shampoo compositions, may comprise a nonionic surfactant. Nonionic surfactants may include those compounds produced by condensation of alkylene oxide groups, hydrophilic in nature, with an organic hydrophobic compound, which may be aliphatic or alkyl aromatic in nature.

Nonlimiting examples of nonionic surfactants, e.g. for use in the shampoo compositions may include the following: (1) polyethylene oxide condensates of alkyl phenols, e.g., the condensation products of alkyl phenols having an alkyl group containing from about 6 to about 20 carbon atoms in either a straight chain or branched chain configuration, with ethylene oxide, the said ethylene oxide being present in amounts equal to from about 10 to about 60 moles of ethylene oxide per mole of alkyl phenol; (2) those derived from the condensation of ethylene oxide with the product resulting from the reaction of propylene oxide and ethylene diamine products; (3) condensation products of aliphatic alcohols having from about 8 to about 18 carbon atoms, in either straight chain or branched chain configurations, with ethylene oxide, e.g., a coconut alcohol ethylene oxide condensate having from about 10 to about 30 moles of ethylene oxide per mole of coconut alcohol, the coconut alcohol fraction having from about 10 to about 14 carbon atoms; (4) long chain tertiary amine oxides of the formula [R¹R²R³N 0] where R¹ contains an alkyl, alkenyl or monohydroxy alkyl radical of from about 8 to about 18 carbon atoms, from 0 to about 10 ethylene oxide moieties, and from 0 to about 1 glyceryl moiety, and R² and R³ contain from about 1 to about 3 carbon atoms and from 0 to about 1 hydroxy group, e.g., methyl, ethyl, propyl, hydroxyethyl, or hydroxypropyl radicals; (5) long chain tertiary phosphine oxides of the formula [RR′R″P(R)0] where R contains an alkyl, alkenyl or monohydroxyalkyl radical ranging from about 8 to about 18 carbon atoms in chain length, from 0 to about 10 ethylene oxide moieties and from 0 to 1 glyceryl moieties and R′ and R″ are each alkyl or monohydroxyalkyl groups containing from about 1 to about 3 carbon atoms; (6) long chain dialkyl sulfoxides containing one short chain alkyl or hydroxy alkyl radical of from 1 to about 3 carbon atoms (usually methyl) and one long hydrophobic chain which include alkyl, alkenyl, hydroxy alkyl, or keto alkyl radicals containing from about 8 to about 20 carbon atoms, from 0 to about 10 ethylene oxide moieties and from 0 to 1 glyceryl moieties; (7) alkyl polysaccharide (APS) surfactants (e.g. alkyl polyglycosides), examples of which are described in U.S. Pat. No. 4,565,647, which is incorporated herein by reference in its entirety, and which discloses APS surfactants having a hydrophobic group with about 6 to about 30 carbon atoms and a polysaccharide (e.g., polyglycoside) as the hydrophilic group; optionally, there can be a polyalkylene-oxide group joining the hydrophobic and hydrophilic moieties; and the alkyl group (i.e., the hydrophobic moiety) can be saturated or unsaturated, branched or unbranched, and unsubstituted or substituted (e.g., with hydroxy or cyclic rings); and (8) polyoxyethylene alkyl ethers, having a general formula RO(CH₂CH₂)ₙH), and polyethylene glycol (PEG) glyceryl fatty esters, having a general formula R(0)0CH₂CH(OH)CH₂(OCH₂CH₂)ₙ0H), wherein n is from 1 to about 200, preferably from about 20 to about 100, and R is an alkyl having from about 8 to about 22 carbon atoms.

Certain nonionic surfactants can also function as foam stabilizers, viscosity control agents, or conditioning agents. Where included, the composition may contain about 0.5 wt percent to about 5.0 wt percent nonionic surfactant, or about 0.75 wt percent to about 2.0 wt percent. Non limiting examples of other anionic, amphoteric/zwitterionic, nonionic, or optional additional surfactants suitable for use in the compositions are described in U.S. Pat. Nos. 3,929,678, 2,658,072; 2,438,091; 2,528,378, which are incorporated herein by reference in their entirety.

### Conditioning Agent

In one embodiment of the present invention, the compositions may comprise one or more conditioning agents. Conditioning agents include materials that are used to give a particular conditioning benefit to hair, nails and/or skin of a human or animal. The conditioning agents useful in the compositions of the present invention typically comprise a water-insoluble, water-dispersible, non-volatile, liquid that forms emulsified, liquid particles. Suitable conditioning agents for use in the composition may be those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix. In an embodiment, one or more conditioning agents are present from about 0.01 wt percent to about 10 wt percent, from about 0.1 wt percent to about 8 wt percent, and from about 0.2 wt percent to about 4 wt percent, by weight of the entire composition.

### Emulsifiers

A variety of anionic emulsifiers can be used in the composition, in particular the shampoo composition, of the present invention as described below. The anionic emulsifiers include, by way of illustrating and not limitation, water-soluble salts of alkyl sulfates, alkyl ether sulfates, alkyl isothionates, alkyl carboxylates, alkyl sulfosuccinates, alkyl succinamates, alkyl sulfate salts such as sodium dodecyl sulfate, alkyl sarcosinates, alkyl derivatives of protein hydrolyzates, acyl aspartates, alkyl or alkyl ether or alkyl aryl ether phosphate esters, sodium dodecyl sulphate, phospholipids or lecithin, or soaps, sodium, potassium or ammonium stearate, oleate or palmitate, alkylarylsulfonic acid salts such as sodium dodecylbenzenesulfonate, sodium dialkylsulfosuccinates, dioctyl sulfosuccinate, sodium dilaurylsulfosuccinate, poly(styrene sulfonate) sodium salt, isobutylene-maleic anhydride copolymer, gum arabic, sodium alginate, carboxymethylcellulose, cellulose sulfate and pectin, poly(styrene sulfonate), isobutylene-maleic anhydride copolymer, gum arabic, carrageenan, sodium alginate, pectic acid, tragacanth gum, almond gum and agar; semi-synthetic polymers such as carboxymethyl cellulose, sulfated cellulose, sulfated methylcellulose, carboxymethyl starch, phosphated starch, lignin sulfonic acid; and synthetic polymers such as maleic anhydride copolymers (including hydrolyzates thereof), polyacrylic acid, polymethacrylic acid, acrylic acid butyl acrylate copolymer or crotonic acid homopolymers and copolymers, vinylbenzenesulfonic acid or 2-acrylamido-2-methylpropanesulfonic acid homopolymers and copolymers, and partial amide or partial ester of such polymers and copolymers, carboxymodified polyvinyl alcohol, sulfonic acid-modified polyvinyl alcohol and phosphoric acid-modified polyvinyl alcohol, phosphated or sulfated tristyrylphenol ethoxylates.

In addition, anionic emulsifiers that have acrylate functionality may also be used in the present composition, such as the present shampoo compositions. Anionic emulsifiers useful herein include, but aren't limited to: poly(meth)acrylic acid; copolymers of (meth)acrylic acids and its (meth)acrylates with Cl-22 alkyl, C1-C8 alkyl, butyl; copolymers of (meth)acrylic acids and (meth)acrylamide; carboxyvinylpolymer; acrylate copolymers such as acrylate/C 10-30 alkyl acrylate crosspolymer, acrylic acid/vinyl ester copolymer/acrylates/vinyl Isodecanoate crosspolymer, acrylates/palmeth-25 acrylate copolymer, acrylate/steareth-20 itaconate copolymer, and acrylate/celeth-20 itaconate copolymer; polystyrene sulphonate, copolymers of methacrylic acid and acrylamidomethylpropane sulfonic acid, and copolymers of acrylic acid and acrylamidomethylpropane sulfonic acid; carboxymethycellulose; carboxy guar; copolymers of ethylene and maleic acid; and acrylate silicone polymer. In an embodiment, the emulsifier, when present, ranges from about 0.01 wt percent to about 5 wt percent, by weight of the entire composition, or from about 0.1 wt percent to about 4 wt percent, or from about 0.1 wt percent to about 3 wt percent. Wt percent is based on the weight of the entire composition. Carbomers can be used for hydrogels in low concentrations for liquid gels and in higher concentrations for solid gels.

### Optional Opacifiers

Some compositions of the present invention may be provided as opacified formulations by incorporating materials therein to achieve a cosmetically attractive pearl-like appearance, known as pearlescence. The opacifying or pearlescent materials may include, but are not limited to, titanium dioxide coated mica, iron oxide coated mica, ethylene glycol mono-stearate, ethylene glycol distearate, polyethylene glycol distearate, bismuth oxychloride coated mica, myristyl myristate, guanine, glitter (polyester or metallic), and mixtures thereof. Other pearlescent materials can be found in U.S. Pat. Nos. 4,654,207 and 5,019,376, herein incorporated by reference. In an embodiment, the concentration of the opacifier, when present, ranges from about 0.01 wt percent to about 5 wt percent, by weight of the entire, or from about 0.1 wt percent to about 3 wt percent, or from about 0.1 wt percent to about 2 wt percent. Wt percent is based on the weight of the entire composition.

### Thickeners

Thickeners or rheology modifiers include, but are not limited to, acrylamide/ammonium acrylate copolymer (and) polyisobutene (and) polysorbate 20; acrylamide/sodium acryloyldimethyl taurate copolymer/isohexadecane/polysorbate 80; acrylates copolymer; acrylates/beheneth-25 methacrylate copolymer; acrylates/C 10-C30 alkyl acrylate crosspolymer; acrylates/steareth-20 itaconate copolymer; ammonium polyacrylate/Isohexadecane/PEG-40 castor oil; Cl 2-16 alkyl PEG-2 hydroxypropyl hydroxyethyl ethylcellulose ((HM-EHEC); carbomer; crosslinked polyvinylpyrrolidone (PVP); dibenzylidene sorbitol; hydroxyethyl ethylcellulose (EHEC); hydroxypropyl methylcellulose (HPMC); hydroxypropyl methylcellulose (HPMC); hydroxypropylcellulose (HPC); methylcellulose (MC); methyl hydroxyethyl cellulose (MEHEC); PEG-150/decyl alcohol/SMDI copolymer; PEG-150/stearyl alcohol/SMDI copolymer; polyacrylamide/C 13-14 isoparaffin/laureth-7; polyacrylate 13/polyisobutene/polysorbate 20; polyacrylate crosspolymer-6; polyamide-3; polyquatemium-37 (and) hydrogenated polydecene (and) trideceth-6; polyurethane-39; sodium acrylate/acryloyl dimethyltaurate/dimethylacrylamide; crosspolymer (and) isohexadecane (and) polysorbate 60; sodium polyacrylate, and combinations thereof. In an embodiment, the concentration of the rheology modifier, when present, ranges from about 0.01 wt percent to about 7 wt percent, by weight of the entire composition, or from about 0.1 wt percent to about 5 wt percent, or from about 0.2 wt percent to about 4 wt percent. Wt percent is based on the weight of the entire composition.

### Buffers

The composition according to the present invention may have a pH ranging from about 3.0 to about 6.5, which may be stabilized by the presence of a buffer system. Suitable buffer solutions can be prepared using, for example, weak acid or weak base systems using citric acid, phosphoric acid, phthalic acid, acetic acid, lactic acid, glycine or mixtures thereof. In each case the proper buffering capacity is obtained by adjusting the final pH of the compositions to within the pH range indicated above. This may be done by using an acid (e.g., HCl, citric acid) or a base (e.g., NaOH, sodium citrate) as may be needed. The amount of buffer employed in the present compositions may depend on the particular acid chosen but is generally from about 0.1 wt percent to about 10 wt percent, preferably from about 0.2 wt percent to about 5 wt percent. Wt percent is based on the weight of the entire composition.

Additional agents, such as benefit agents, may also be included in the composition according to the present invention. The benefit agent may comprise a material selected from the group consisting of prebiotics; perfumes; brighteners; enzymes; sensates (cooling or warming); attractants, preservatives; dyes; pigments; bleaches; and mixtures thereof.

It should be further appreciated that the inventive combination of natural actives disclosed herein may also be used in combination with secondary benefit actives, such as secondary scalp benefit actives, e.g. soluble and/or insoluble secondary actives. Such secondary actives may include but are not limited to azoles, such as ketoconazole, econazole, climbazole, and elubiol; keratolytic agents such as salicylic acid; and zinc-containing layered (ZLM) materials, pyridinethione anti-dandruff particulates such as zinc pyrithione, coal tar, sulfur, charcoal, whitfield's ointment, castellani's paint, aluminum chloride, gentian violet, octopirox (piroctone olamine), ciclopirox olamine, undecylenic acid and it's metal salts, potassium permanganate, selenium sulphide, sodium thiosulfate, propylene glycol, urea preparations, griseofulvin, 8-Hydroxyquinoline ciloquinol, thiobendazole, thiocarbamates, haloprogin, polyenes, hydroxypyridone, morpholine, benzylamine, allylamines (such as terbinafine), Sensiva SC-50, Elestab HP-100, azelaic acid, lyticase, iodopropynyl butylcarbamate (IPBC), isothiazalinones such as octyl isothiazalinone, other natural oils, extracts, or compounds such as oil of bitter orange, tea tree oil, clove leaf oil, coriander, palmarosa, berberine, thyme red, cinnamon oil, cinnamic aldehyde, citronellic acid, hinokitol, ichthyol pale, and combinations thereof. In an embodiment, the concentration of the secondary benefit agent may range from about 0.01 wt percent to about 5 wt percent, by weight of the entire composition, or from about 0.1 wt percent to about 3 wt percent, or from about 0.1 wt percent to about 2 wt percent. Wt percent is based on the weight of the entire composition. In an alternative embodiment of the present invention, the inventive combination of natural actives disclosed herein may also be void of any of the foregoing recited secondary actives.

Preferably the at least one prebiotic compound may be comprised as additional agent in the composition of the invention, i.e. as other ingredient. In a very broad concept, prebiotics are all those compounds which can be metabolized by probiotics. Preferably prebiotics are non-digestible or poorly digestible by a mammal. Prebiotics are well known in the art and when used in the present invention there is no particular limitation of the prebiotic as such.

Preferably, the at least one prebiotic product in the composition may be selected from the following compounds and compositions: non-digestible carbohydrates, beta-glucans, mannan-oligosaccharides, inulin, human oligofructose, milk oligosaccharides (HMO), galactooligosaccharides (GOS), lactulose, lactosucrose, galactotriose, fructo-oligosaccaride (FOS), cellobiose, cellodextrins, cylodextrins, maltitol, lactitol, glycosilsucrose. Mannan-oligosaccharides and/or inulin may be preferred.

HMOs may include lacto-N-tetraose, lacto-N-fucopentaose, lacto-N-triose, 3 ‘-sialyllactose, lacto-N-neofucopentaose, sialic acid, L-fucose, 2-fucosyllactose, 6’-sialyllactose, lacto-N-neotetraose and 3-fucosyllactose.

D- and L-fucose may be suitable and may strengthen natural defense of skin, stimulate epidermis immune defense and/or prevent and/or treat cutaneous autoimmune disease. In a preferred embodiment of the invention the composition comprises D- and/or L-fucose.

In one preferred embodiment of the invention the composition further comprises L-fucose in a concentration in the composition of 10 mM to 500 mM.

The composition according to the present invention may generally be prepared by conventional methods known in the art, e.g. of preparing a shampoo compositions, a crème, an oil an emulsion or the like. Such methods typically involve mixing of the ingredients in one or more steps to a relatively uniform state, with or without heating, cooling, application of vacuum, and the like. The compositions may be prepared such as to optimize stability (physical stability, chemical stability, photostability) and/or delivery of the active. This optimization may include appropriate pH, and exclusion of materials that can complex or react with the active agent(s) and thus negatively impact stability or delivery.

The composition may be in a single phase or a single product, or the composition may be in a separate phases or separate products. If two products are used, the products may be used together, at the same time or sequentially. Sequential use may occur in a short time period, such as immediately after the use of one product, or it may occur over a period of hours or days.

In one embodiment of the invention the composition is a leave-on composition comprising viable probiotic bacteria in a concentration from 10³ to 10¹³ colony forming units of bacteria per gram. More specifically a composition comprising 10⁴ to 10¹⁰ colony forming units of bacteria per gram, more specifically a composition comprising 10⁵ to 10⁹ colony forming units of bacteria per gram.

In one embodiment of the invention the composition is a leave-on composition comprising viable probiotic bacteria in a concentration of at least 10³ colony forming units of bacteria per gram. More specifically a composition comprising of at least 10⁴ colony forming units of bacteria per gram, more specifically a composition comprising at least 10⁵ colony forming units of bacteria per gram, more specifically a composition comprising at least 10⁶ colony forming units of bacteria per gram, more specifically a composition comprising at least 10⁷ colony forming units of bacteria per gram.

In one embodiment of the invention the composition comprises a postbiotic fraction of actives, such as the anti-dandruff actives or the anti-mycosis actives.

Use of the composition according to the present invention as an anti-dandruff composition: According to an embodiment of the present invention, a method is provided for the treatment of a subject having dandruff and/or to prevent or inhibit the onset of dandruff symptoms associated with the proliferation of yeasts of the Malassezia genus on a scalp of a subject. The method includes contacting the subject's scalp or keratinous tissue with an effective quantity of the anti-dandruff composition of the present invention. The anti-dandruff composition may be massaged onto the scalp and should remain in contact with the subjects scalp or keratinous tissue for a duration of at least 15 seconds or more. Depending on the formulation, the anti-dandruff composition may be a leave in or it may be rinsed out.

Accordingly, in another embodiment, the method comprises topically applying an anti-dandruff composition comprising an effective amount of the anti-dandruff actives to a region of the subject's skin where inhibition of Malassezia is needed or wanted, where the anti-dandruff actives remain in contact with the region either as a leave-on composition or for a duration of 15 seconds or more; and then rinsed out. In still another embodiment, the method comprises applying the composition according to a regimen, wherein said regimen comprises: (a) cleansing the scalp to form a cleansed scalp; (b) topically applying the anti-dandruff composition to said cleansed scalp.

Use of the composition according to the present invention as an anti-mycosis composition: According to an embodiment of the present invention, a method is provided for the treatment of a subject having mycosis on the nails or on the feet, and/or to prevent or inhibit the onset of mycosis symptoms associated with the proliferation of the Malassezia genus on the nails and/or the feet of a subject. The method includes contacting the subject's nails and/or feet with an effective quantity of the anti-mycosis composition of the present invention. The anti-mycosis composition may be massaged onto the nails and/or feet and should remain in contact with the subjects nails and/or feet for a duration of at least 15 seconds or more. Depending on the formulation, the anti-mycosis composition may be a leave-on or it may be rinsed-out.

Accordingly, in another embodiment, the method comprises topically applying an anti-mycosis composition comprising an effective amount of the anti-mycosis actives to a region of the subject's nails and/or feet where inhibition of fungi is needed or wanted, where the anti-mycosis actives remain in contact with the region either as a leave-on composition or for a duration of 15 seconds or more; and then rinsed out. In still another embodiment, the method comprises applying the composition according to a regimen, wherein said regimen comprises: (a) cleansing the scalp to form a cleansed nail or foot; (b) topically applying the anti-mycosis composition to said cleansed nails or feet.

According to the present invention fungal infections may include dandruff and/or mycosis. Mycosis may relate to any disease caused by fungi and may affect different parts of the body, in particular the feet, ears, hands and/or the nails of the human or animal body.

A preferred embodiment of the present invention relates to a composition according to the present invention for use in the prevention and/or treatment of mycoses in a human or in an animal.

Preferably, the prevention and/or treatment of mycoses and/or dandruff in a human or in an animal may include prevention and/or treatment of conditions associated with the proliferation of yeasts of the Malassezia genus on a scalp, on hands, on ears, on feet, on nails or on skin of a subject.

In a further embodiment of the present invention the composition comprising one or more probiotic bacterial strains (preferably an isolated probiotic bacterial strain) and/or one or more active of the one or more probiotic bacterial strains (preferably an isolated probiotic bacterial strain) for use in the prevention and/or treatment of mycoses in a human or in an animal.

The prevention and/or treatment of mycoses in a human or in an animal may be prevention and/or treatment of:
dandruff conditions associated with the proliferation of yeasts of the Malassezia genus on a scalp or skin of a human or an animal; or
fungal nail infection associated with the proliferation of fungi or yeasts, e.g. of the Tinea unguium or Trichophyton spp. on the nails if a human or an animal; or
In an embodiment of the present invention the composition comprises a nucleic acid and/or a nucleotide.

In a further embodiment of the present invention the composition comprises no plant material and/or fibre material. Preferable the fibre material is plant fibre material

Preferably the content of fibre material in the composition is less than 5% (w/w) relative to the entire composition, such as less than 4% (w/w), e.g. less than 3% (w/w), such as less than 2% (w/w), e.g. less than 1% (w/w), such as less than 0.5 (w/w), e.g. less than 0.1% (w/w), such as 0%.

In an embodiment of the present invention the composition comprises two or more actives of the one or more isolated probiotic bacterial strains (the specific actives may be as described above), such as 3 or more, e.g. 4 or more, such as 5 or more, e.g. 4 or more, such as 10 or more, e.g. 15 or more, such as 20 or more, e.g. 25 or more.

The subject according to the present invention may be a mammal selected from humans, dogs, cats, horses, cattle or sheeps.

The composition according to the present invention may be used daily, weekly, or in a variety of regimens. The composition may be used more than once a day, such as at night and in the morning. The composition may be used after washing the hair (also on wet or dry hair), or after washing the body, feet or nails, which may mean using the composition more than once per day on certain days or use only a few times per week. The composition may be used three times per day, twice per day, once per day, six times per week, five times per week, four times per week, three times per week, two times per week, or one time per week. In some embodiments, the anti-dandruff composition is used four, five, six or seven times per week.

According to another embodiment, the composition may be applied to at least once a day for at least about one week, or at least twice a day for at least about one week.

According to another embodiment, the composition may be applied to at least once a day for at least about four weeks, or at least twice a day for at least about four weeks. According to another embodiment, the composition may be applied at least once a day for at least about eight weeks.

The composition may be used by males and females. The composition may be used by mammals of any age, including newborn, infants, babies and kids.

In an embodiment of the present invention invention the composition may be an anti-dandruff composition. Preferably, the anti-dandruff composition may be used for prevention or treatment of cradle cap.

In another embodiment of the present invention the composition may be applied to the skin, fur, oral cavity, genital area, ears and/or body orifices of a human or an animal.

Preferably, the composition according to the present invention may be applied topical to an animal. In a further preferred embodiment, the animal may be selected from dogs, cats, cattle, cows, sheep, goats, horses and birds.

the present invention may relate to a composition (such as an anti-dandruff composition and/or an anti-mycosis composition) comprising a probiotic microorganism able to produce actives when applied topically on infected area, like the scalp, the hands, skin, ears, the feet and/or the nails.

The present invention may relate to an anti-dandruff composition comprising a probiotic microorganism able to produce anti-dandruff actives when applied topically on scalp.

Preferably, the composition comprises at least 2 anti-dandruff actives produced by a probiotic microorganism selected from; bacteriocins, lactic acid, acetic acid, succinic acid, azelaic acid, salicylic acid, indole-3-lactic acid, indole-3-acetic acid, 2-hydroxybuturic acid, N-acetyl tryptophan, glycolic acid, N-acetyl glutamin and N-acetylaspartic acid.

Composition for treatment of dandruff and/or mycosis wherein the composition comprises viable probiotic microorganisms in at least a concentration of 10³ colony forming unit per gram of composition wherein the probiotic microorganism is able to produce anti-dandruff and/or anti-mycosis actives.

Composition with a pH below 6.5 for treatment of dandruff and/or mycosis wherein the composition comprises viable probiotic microorganisms in at least a concentration of 10³ colony forming unit per gram of composition wherein the probiotic microorganism is able to produce anti-dandruff and/or anti-mycosis actives.

A composition comprising a bacteriocin producing probiotic microorganism wherein same microorgnaism produces at least two of the following metabolites; succinic acid, azelaic acid, salicylic acid, 2-Hydroxyisocaproic acid, indole-3-lactic acid, indole-3-acetic acid, 2-hydroxybuturic acid, N-acetyl tryptophan, glycolic acid, N-acetyl-glutamine and N-acetylaspartic acid.

Composition comprising a postbiotic fraction from a lactic acid bacteria able to produce at least two of the following anti-dandruff and/or anti-mycosis actives: succinic acid, azelaic acid, salicylic acid, 2-Hydroxyisocaproic acid, indole-3-lactic acid, indole-3-acetic acid, 2-hydroxybuturic acid, N-acetyl tryptophan, glycolic acid, N-acetyl glutamine or N-acetylaspartic acid.

Anti-dandruff and/or anti-mycosis composition comprising indole-3-lactic acid wherein the indole-3-lactic acid is produced by a lactic acid bacteria.

Anti-dandruff and/or anti-mycosis composition comprising salicylic acid wherein the salicylic acid is produced by a lactic acid bacteria.

Anti-dandruff and/or anti-mycosis composition comprising gluconic acid wherein the gluconic acid is produced by a lactic acid bacteria.

Anti-dandruff and/or anti-mycosis composition comprising phenyl lactic acid wherein the phenyl lactic acid is produced by a lactic acid bacteria.

Anti-dandruff and/or anti-mycosis composition comprising 2-Hydroxyisocaproic acid wherein the 2-Hydroxyisocaproic acid is produced by a lactic acid bacteria.

Anti-dandruff and/or anti-mycosis composition comprising 2-hydroxy-buturic acid wherein the 2-hydroxy-buturic acid is produced by a lactic acid bacteria.

Anti-dandruff and/or anti-mycosis composition comprising N-acetylaspartic acid wherein the N-acetylaspartic acid is produced by a lactic acid bacteria.

Anti-dandruff and/or anti-mycosis composition comprising azelaic acid wherein the azelaic acid is produced by a lactic acid bacteria.

Anti-dandruff and/or anti-mycosis composition comprising succinic acid wherein the succinic acid is produced by a lactic acid bacteria.

Anti-dandruff and/or anti-mycosis composition comprising indole 3-acetic acid wherein the indole 3-acetic acid is produced by a lactic acid bacteria.

Anti-dandruff and/or anti-mycosis composition comprising 3-phenyllactic acid wherein the 3-phenyllactic acid is produced by a lactic acid bacteria.

Anti-dandruff and/or anti-mycosis composition comprising N-acetylglutamine wherein the N-acetylglutamine is produced by a lactic acid bacteria.

Anti-dandruff and/or anti-mycosis composition comprising 2-Hydroxyisocaproic acid and salicylic acid produced by a lactic acid bacteria.

Anti-dandruff and/or anti-mycosis composition according to any one of the compositions described above wherein the composition comprises at least one bacteriocin.

Anti-dandruff and/or anti-mycosis composition comprising bacteriocin, salicylic acid, indole-3-lactic acid, and 3-phenyllactic acid.

Anti-dandruff and/or anti-mycosis composition comprising bacteriocin, 2-Hydroxyisocaproic acid, salicylic acid, indole-3-lactic acid, 2-hydroxybuturic acid and N-acetylaspartic acid

Anti-dandruff and/or anti-mycosis composition according to any of the compositions described above wherein the composition is produced by an isolated lactic acid bacteria.

Anti-dandruff and/or anti-mycosis composition according to any of the compositions described above further comprising at least one antimicrobial bacterial metabolite chosen from a group comprising hydrogen peroxide, 3-phenyllactic acid, 3-hydroxyphenyllactic acid, 4-hydroxyphenylactic acid, 2-Hydroxyisocaproic acid, 3-hydroxy propanaldehyde, 1,2 propandiol, 1,3 propandiol, succinic acid, ethanol, acetic acid, carbonic acid, propanoic acid, butyric acid, cyclic dipeptides, cyclo(L-Phe-L-Pro), cyclo(L P-Traps-4-OH-L-Pro), 3-(R)-hydroxydecanoic acid, 3-hydroxy-5-cic dodecanoic acid, 3-(R)-hydroxy dodecanoic acid, and 3-(R)-hyroxytetradecanoic acid.

Anti-dandruff and/or anti-mycosis composition according to any of the compositions described above, wherein the probiotic microorganism is selected from the group; Lactiplantibacillus plantarum LB356R (DSM 33094), Lactiplantibacillus plantarum LB244R (DSM 32996), Weissella viridescens LB10G (DSM 32906), Lacticaseibacillus paracasei LB113R (DSM 32907), Lacticaseibacillus paracasei LB116R (DSM 32908), Levilactobacillus brevis LB152G (DSM 32995), Lacticaseibacillus paracasei LB28R (DSM 32994), Enterococcus faecium LB276R (DSM 32997), Leuconostoc mesenteriodes LB349R (DSM 33093), Lactiplantibacillus plantarum LB316R (DSM 33091), Lactiplantibacillus plantarum LB312R (DSM 33098), Pediococcus pentosaceus LB606R (DSM 33730), Lactiplantibacillus plantarum LB679R (DSM 33731), Lactobacillus crispatus LB714R (DSM 33732); Lactobacillus gasseri LB905R (DSM 34094), Lactobacillus crispatus LB912R (DSM 34095), Lactobacillus jensenii LB918R (DSM 34096), Lactobacillus crispatus LB919R (DSM 34097), Lacticaseibacillus paracasei subsp. paracasei LB555R (DSM 34249), Lactiplantibacillus plantarum LB681R (DSM 34250); and/or any combinations hereof.

### Deposit of Biological Material

The lactic acid bacteria according to the present invention include in particular microorganisms or analogs, fragments, derivatives, ferments, lysates, mutants or combinations thereof selected from the group comprising:
the following microorganisms deposited on 5th of May 2022 with the German Collection for Microorganisms and Cell Cultures: Lactiplantibacillus plantarum LB681R (DSM 34250), and Lacticaseibacillus paracasei subsp. paracasei LB555R (DSM 34249);
the following microorganisms deposited on 14th of December 2020 with the German Collection for Microorganisms and Cell Cultures: Lactobacillus crispatus LB714R (DSM 33732), Pediococcus pentosaceus LB606R (DSM 33730), and/or Lactiplantibacillus plantarum LB679R (DSM 33731);
the following microorganisms deposited 25 Nov. 2021: Lactobacillus crispatus LB919R (DSM 34097), Lactobacillus crispatus LB912R (DSM 34095), Lactobacillus gasseri LB905R (DSM 34094), and/or Lactobacillus jensenii LB918R (DSM 34096);
the following microorganisms deposited on 10th of April 2019 with the German Collection for Microorganisms and Cell Cultures: Lactobacillus plantarum LB356R (DSM 33094), Leuconostoc mesenteriodes LB349R (DSM 33093), Lacticaseibacillus paracasei LB316R (DSM 33091), and/or Lactiplantibacillus plantarum LB312R (DSM 33098);
the following microorganisms deposited on 13th of December 2018 with the German Collection for Microorganisms and Cell Cultures: Lactobacillus plantarum LB244R (DSM 32996), Enterococcus faecium LB276R (DSM 32997), Levilactobacillus brevis LB152G (DSM 32995), and/or Lacticaseibacillus paracasei LB28R (DSM 32994); and/or
the following microorganisms deposited on 28th of August 2018 with the German Collection for Microorganisms and Cell Cultures: Lactobacillus plantarum LB116R (DSM 32908), Lacticaseibacillus paracasei LB113R (DSM 32907), and/or Weissella viridescens LB10G (DSM 32906).

All patent and non-patent references cited in the present application, are hereby incorporated by reference in their entirety.

The invention will now be described in further details in the following non-limiting examples.

### EXAMPLES

### Example 1

Malassezia furfur with CCUG number 59937 was delivered from “Culture Collection University Gothenburg”. The strain was isolated from a 15-year-old girl with human pityriasis versicolor. M. furfur 59937 was inoculated in potato dextrose broth (Sigma) and incubated for five days at 30° C. After incubation, the culture was streaked with a cotton stick on potato dextrose agar (Sigma) plates to create a lawn. On top of the streaked agar plates, 20 μL of 48 hours cultures of Lactiplantibacillus plantarum LB244R® (DSM 32996), Lactiplantibacillus plantarum LB356R® (DSM 33094) and Pediococcus pentosaceus LB606R® (DSM 33730) were spotted. The plates were incubated for five days at 30° C. After incubation, the plates were visually inspected. Around all spots of lactic acid bacteria, a +1 cm inhibition zone in the M. furfur lawn could be observed.

### Example 2

### Different Formulations

### Anti-Dandruff Shampoo Compositions:

Shampoo Formulation A11

### Ingredients:

Water 65% (w/w)
Postbiotic (Lactobio Lactiplantibacillus LB356R® ferment-lysate) 15% (w/w)—comprising 95-98% (w/w) moisture (equals to a cell concentration of 10⁷-10¹⁰ cells per ml)
Lauroyl/Myristoyl methyl glucamide 7% (w/w)
Decyl Glucoside 2% (w/w)
Betaine 2% (w/w)
Sodium cocoyl isethionate 1% (w/w)
Glycerin 1% (w/w)
Cocamide mipa 1% (w/w)
Hydrogenated coconut acid 1% (w/w)
Sorbic acid 1% (w/w)
Sodium isethionate 1% (w/w)
Sodium phytate 1% (w/w)
Ethanol 1% (w/w)
Sodium benzoate 0.5% (w/w)
Potassium sorbate 0.5% (w/w)
PH adjusted to 4.5

All ingredients except the probiotic microorganism is heated until melting, cooled with agitation and the probiotic microorganism is added as postbiotic solution when the shampoo is cooled to 20° C.

### Shampoo Formulation A12:

### Ingredients:

Water 39% (w/w)
Postbiotic (Lactiplantibacillus plantarum LB356R® ferment-lysate) 14% (w/w)-comprising 95-98% (w/w) moisture (equals to a cell concentration of 10⁷-10¹⁰ cells per ml)
Sodium Laureth Sulfate 13% (w/w)
Disodium Cocoamphodiacetate 8% (w/w)
PEG-200 Hydrogenated Glyceryl Palmitate 5% (w/w)
Polysorbate 20 5% (w/w)
Hexylene Glycol 2% (w/w)
Disodium Ricinoleamido MEA 2% (w/w)
Sulfosuccinate 2% (w/w)
Sodium Benzoate 1% (w/w)
Polysorbate 21 1% (w/w)
Sodium Methylparaben 1% (w/w)
Polyquaternium-10 1% (w/w)
PEG-7 Glyceryl Cocoate, Menthol 1% (w/w)
Ethylparaben 1% (w/w)
Propylene Glycol 1% (w/w)
Butylphenyl Methylpropional 1% (w/w)
Benzyl Alcohol 1% (w/w)
Sodium Hydroxide 1% (w/w)
pH adjusted to 3.7

All ingredients except the probiotic microorganism is heated until melting, cooled with agitation and the probiotic microorganism is added as postbiotic solution when the shampoo is cooled to 20° C.

### Shampoo Formulation A13

### Ingredients:

Water 21% (w/w)
sodium coco-sulfate 20% (w/w)
coco-glucoside 20% (w/w)
glycerin 12% (w/w)
sodium cocoyl isethionate 6% (w/w)
lauryl glucoside 5% (w/w)
stearyl citrate 4% (w/w)
Vitis vinifera (grape/raisin) seed oil 4% (w/w)
citric acid 3% (w/w)
Lactiplantibacillus plantarum LB244R® 3% (w/w) (lyophilized powder with the concentration of 9×10¹⁰ CFU/g)
panthenol (vitamin B5) 0.5% (w/w)
glyceryl oleate 0.5% (w/w)
inulin 0.5% (w/w)
potassium sorbate 0.5% (w/w)

All ingredients except the probiotic microorganism is heated until melting, cooled with agitation, pH adjusted to 5.0 and the probiotic microorganism is added when the shampoo is cooled to 20° C.

### Shampoo Formulation A14

### Ingredients:

Water 40% (w/w)
Sodium C14-16 Olefin Sulfonate 18% (w/w)
Cocamidopropyl Betaine 8% (w/w)
Sodium Chloride 4% (w/w)
Cocamidopropyl Hydroxysultaine 4% (w/w)
Cocos Nucifera (Coconut) Oil 5%
PPG-2 Hydroxyethyl Coco/Isostearamide 2%
Acrylates Copolymer 2%
Inulin 2%
Lactiplantibacillus plantarum LB356R® 2% (w/w) (lyophilized powder with the concentration of 2×10¹⁰ CFU/g)
Hydrolyzed Milk Protein 2%
Albumen 2%
Amodimethicone 1%
Sodium Cocoyl Isethionate 1%
Glycol Distearate 1%
Laureth-4 1%
Polyquaternium-6 1%
Polyquaternium-10 1%
Trideceth-12 1%
Cetrimonium Chloride 0.5%
Guar Hydroxypropyltrimonium Chloride 0.5%
PEG-120 Methyl Glucose Dioleate 0.5%
Disodium EDTA 0.4%
Diazolidinyl Urea 0.1%

The freeze dried probiotic microorganism is capsulated in a dual chamber plastic cap comprising 2 g of freeze dried material (AccuRec kit bi-phase cap obtained from Bormioli Pharma, Italy). All ingredients except the probiotic microorganism is heated until melting, pH measured to 4.8, cooled with agitation and 100 ml bottles are filled with the composition and closed with the cap comprising the probiotic microorganism in a separated compartment. Before use, the microorganisms are released from the cap into the bottle and shaken well.

### Dry Shampoo Loose Powder; Leave-In Formulation B11

Zea Mays Starch 46% (w/w)
Aluminum starch octenylsuccinate 38% (w/w)
Kaolin 10% (w/w)
Cyclodextrin 5% (w/w)
Lactiplantibacillus plantarum LB356R® 1% (w/w) (lyophilized powder with the concentration of 2×10¹⁰ CFU/g)

The lyophilized viable probiotic microorganism is mixed with cyclodextrin followed by mixing with the rest of the ingredients at room temperature.

The anti-dandruff composition is to be either sprayed or sprinkled onto the scalp as a leave-on composition.

### Dry Shampoo Loose Powder; Leave-In Formulation B12

Talc 97% (w/w)
Lactiplantibacillus plantarum LB356R® 2% (w/w) (lyophilized powder with the concentration of 2×10¹⁰ CFU/g)
Inulin 1% (w/w)

The lyophilized viable probiotic microorganism is gently blended with talc at room temperature. The anti-dandruff composition is to be either sprayed or sprinkled onto the scalp as a leave-on composition.

### Dry Shampoo Loose Powder; Leave-In Formulation B13

Corn starch 90% (w/w)
Talc 9% (w/w)
Pediococcus pentosaceus LB606R® 1% (w/w) (lyophilized powder with the concentration of 9×10⁹ CFU/g)

The lyophilized viable probiotic microorganism is gently blended with corn starch and talc at room temperature.

The anti-dandruff composition is to be either sprayed or sprinkled onto the scalp as a leave-on composition.

### Dry Shampoo Loose Powder; Leave-In Formulation B14

Potato starch 40% (w/w)
Corn starch 37% (w/w)
Kaolin 10% 8 w/w)
Talc 9% (w/w)
Cyclodextrin 4% (w/w)
Pediococcus pentosaceus LB606R® 1% (w/w) (lyophilized powder with the concentration of 9×10⁹ CFU/g)

The lyophilized viable probiotic microorganism is gently blended with the other ingredients at room temperature.

The anti-dandruff composition is to be either sprayed or sprinkled onto the scalp as a leave-on composition.

### Hair Rinse Leave-In Formulation C11

Water 70% (w/w)
Apple Cider Vinegar 10% (w/w)
Cetrimonium Chloride 5% (w/w)
Glycerin 3% (w/w)
Hydrolyzed Vegetable Protein 1% (w/w)
PG-Propyl Silanetriol 1% (w/w)
Argania Spinosa Kernel Oil 1% (w/w)
Aloe Barbadensis Leaf Juice 1% (w/w)
Macadamia Ternifolia Seed Oil 1% (w/w)
Quaternium-96 1% (w/w)
Polyquaternium-55 1% (w/w)
Dipropylene Glycol 1% (w/w)
Propylene Glycol 1% (w/w)
Diazolidinyl Urea 1% (w/w)
Iodopropynyl Butylcarbamate 1% (w/w)
Pediococcus pentosaceus LB606R® 1% (w/w) (lyophilized powder with the concentration of 9×10⁹ CFU/g)

The freeze dried probiotic microorganism is capsulated in a dual chamber plastic cap comprising 2 g of freeze dried material (AccuRec kit bi-phase cap obtained from Bormioli Pharma, Italy). All ingredients except the probiotic microorganism is heated until melting, pH measured to 4.5, cooled with agitation and 100 ml bottles are filled with the composition and closed with the cap comprising the probiotic microorganism in a separated compartment. Before use, the microorganisms are released from the cap into the bottle and shaken well.

### Hair Rinse Leave-In Formulation C12

Water 70% (w/w)
Apple Cider Vinegar (10% w/w)
Glycerin (3% w/w)
Rapeseedamidopropyl Ethyldimonium Ethosulfate 1% (w/w)
Polyacrylamidopropyltrimonium Chloride 1% (w/w)
Hydrolyzed Vegetable Protein PG-Propyl Silanetriol 1% (w/w)
Argania Spinosa Kernel Oil 1% (w/w)
Aloe Barbadensis Leaf Juice 1% (w/w)
Macadamia Ternifolia Seed Oil 1% (w/w)
Quaternium-96 1% (w/w)
Polyquaternium-55 1% (w/w)
Dipropylene Glycol 1% (w/w)
Propanediol 1% (w/w)
Propylene Glycol 1% (w/w)

Diazolidinyl Urea 1% (w/w)
Tetrasodium Glutamate Diacetate 1% (w/w)
Iodopropynyl Butylcarbamate 1% (w/w)
Pediococcus pentosus LB606R® 2% (w/w) (lyophilized powder with the concentration of 9×10⁹ CFU/g)
Lactiplantibacillus plantarum LB356R® 1% (w/w) (lyophilized powder with the concentration of 2×10¹⁰ CFU/g)

The freeze dried probiotic microorganism is capsulated in a dual chamber plastic cap comprising 2 g of freeze dried material (AccuRec kit bi-phase cap obtained from Bormioli Pharma, Italy). All ingredients except the probiotic microorganism is heated until melting and solution as a homogene solution, pH measured to 4.4, cooled with agitation and 100 ml bottles are filled with the composition and closed with the cap comprising the probiotic microorganism in a separated compartment. Before use, the microorganisms are released from the cap into the bottle and shaken well.

### Hair Rinse Leave-In Formulation C13

Water 70% (w/w)
Lactiplantibacillus plantarum LB356R® ferment lysate (15% w/w)-comprising 95-98% (w/w) moisture (equals to a cell concentration of 10⁷-10¹⁰ cells per ml)
Glycerin (3% w/w)
Rapeseed amidopropyl Ethyldimonium Ethosulfate 1% (w/w)
Polyacrylamidopropyltrimonium Chloride 1% (w/w)
Argania Spinosa Kernel Oil 1% (w/w)
Aloe Barbadensis Leaf Juice 1% (w/w)
Macadamia Ternifolia Seed Oil 1% (w/w)
Quaternium-96 1% (w/w)
Polyquaternium-55 1% (w/w)
Dipropylene Glycol 1% (w/w)
Propanediol 1% (w/w)
Propylene Glycol 1% (w/w)
Diazolidinyl Urea 1% (w/w)
Tetrasodium Glutamate Diacetate 1% (w/w)

Gently heated until all ingredients are solubilized. The L. plantarum LB356R® ferment lysate was added after the composition was cooled to room temperature. pH was measured to 4.4.

### Hair Serum Leave-In Formulation C14

Helianthus annuus seed oil
Ricinus communis seed oil
Pediococcus pentosaceus LB606R 1% (w/w) (lyophilized powder with the concentration of 9×10⁹ CFU/g)
Lactiplantibacillus plantarum LB356R® 2% (w/w) (lyophilized powder with the concentration of 2×10¹⁰ CFU/g)
Tocopherol (Vit E)

### Hair Serum Leave-In Formulation C15

Helianthus annuus seed oil 75% (w/w)
Ricinus communis seed oil 22% (w/w)
Lactiplantibacillus plantarum LB356R® 2% (w/w) (lyophilized powder with the concentration of 2×10¹⁰ CFU/g)
Tocopherol (Vit E) (1% w/w)

Formulation C13 and C14 was mixed at room temperature without heating of the ingredients.

Powder compositions are useful not only as dry shampoos but also for scalp treatment, skin treatment and also for diaper rash.

### Topical Leave-on Formulation C16

Helianthus annuus seed oil 55% (w/w)
Ricinus communis seed oil 42% (w/w)
Lactiplantibacillus plantarum LB356R® 2% (w/w) (lyophilized powder with the concentration
of 2×10¹⁰ CFU/g)
Tocopherol (Vit E) (1% w/w)
All ingredients are mixed at room temperature
Topical leave-on formulation C17
Water 83.8% (w/w)
Pediococcus pentosaceus LB606R® ferment lysate (15% w/w)-comprising 95-98% (w/w) moisture (equals to a cell concentration of 10⁷-10¹⁰ cells per ml)
Betaine 1% (w/w)
Carbomer 0.2% (w/w)

Water, betaine and carbomer is mixed until a clear solution is obtained, pH adjusted to pH 4.7 and the ferment lysate is added.

### Example 3

Metabolomics were done on 3 different Lactic acid bacteria. Lactiplantibacillus plantarum LB356R (DSM 33094), Lactiplantibacillus plantarum LB244R (DSM 32996), Pediococcus pentosaceus LB606R (DSM 33730).

The microorganisms were grown in MRS medium for 24 hours at 30° C. The supernatant analysed by the semi-polar metabolites method. Sample analysis was carried out by MS-Omics (Vedbæk, Denmark) as follows.

The samples were diluted 10 times in 10 mM ammonium formate with 0.1% formic acid. LC-MS method

The analysis was carried out using a UPLC system (Vanquish, Thermo Fisher Scientific) coupled with a high-resolution quadrupole-orbitrap mass spectrometer (Q Exactive™ HF Hybrid Quadrupole-Orbitrap, Thermo Fisher Scientific). An electrospray ionization interface was used as ionization source. Analysis was performed in negative and positive ionization mode. A QC sample was analysed in MS/MS mode for identification of compounds. The UPLC was performed using a slightly modified version of the protocol described by Catalin et al. (UPLC/MS Monitoring of Water-Soluble Vitamin Bs in Cell Culture Media in Minutes, Water Application note 2011, 720004042en).

### Data Processing

Data was processed using Compound Discoverer 3.1 (ThermoFisher Scientific) and TraceFinder 4.1 (ThermoFisher Scientific).

### Compound Extraction

One compound often gives rise to a signal in more than one mass trace (due to e.g. naturally occurring C13 isotopes, adducts, and/or fragments) a compound will therefore almost always be represented by more than one feature with the same retention time but different masses. The compound extraction performed by Compound Discoverer consists of the following four steps:
1) First, features are extracted from the raw data.
2) The feature detection is followed by grouping of features belonging to the same compound.
3) This additional information (e.g. isotope pattern) is then used together with the accurate mass to determine the molecular formula.
4) The total information collected for each compound are then used in the following identification step.

The analysis was carried out using a Thermo Scientific Vanquish LC coupled to Thermo Q Exactive HF MS. An electrospray ionization interface was used as ionization source. Analysis was performed in negative and positive ionization mode. The UPLC was performed using a slightly modified version of the protocol described by Catalin et al. (UPLC/MS Monitoring of Water-Soluble Vitamin Bs in Cell Culture Media in Minutes, Water Application note 2011, 720004042en). Peak areas were extracted using Compound Discoverer 3.1 (Thermo Scientific). Identification of compounds were performed at four levels; Level 1: identification by retention times (compared against in-house authentic standards), accurate mass (with an accepted deviation of 3 ppm), and MS/MS spectra, Level 2a: identification by retention times (compared against in-house authentic standards), accurate mass (with an accepted deviation of 3 ppm). Level 2b: identification by accurate mass (with an accepted deviation of 3 ppm), and MS/MS spectra, Level 3: identification by accurate mass alone (with an accepted deviation of 3 ppm).

A total of 1606 compounds were detected in the samples. Hereof were 271 annotated on level 3, 10³ on level 2b, 113 on level 2a, and 60 on level 1.

Lactic acid, acetic acid, succinic acid, azelaic acid, salicylic acid, indole-3-lactic acid, indole-3-acetic acid, 2-hydroxybuturic acid, 2-Hydroxyisocaproic acid and N-acetylaspartic acid were all annotated at level 1 in significant amounts for the strains.

Organic acid was in the concentration above 3% (w/w) for all fermentations.

### Example 4

### Evaluation Test on Dandruff

12 persons with dandruff evaluated composition C14 for 2 weeks.

The leave-in composition was massaged into the affected area of the scalp once every day for 14 days. The composition was left for at least 8 hours before any wash.

Test persons were ask to self-assess the dandruff as compared to the dandruff before initiation of the treatment. Dandruff was assessed according to the following score.

| Self-assessment relative to before treatment | score |
|---|---|
| Dandruff got worse | −1 |
| No change | 0 |
| Dandruff got significant less | +1 |
| Dandruff disappeared | +2 |

### Results

| | Self assess score | Self assess score |
|---|---|---|
| Test person | after 7 days | after 14 days |
| 1 | 2 | 2 |
| 2 | 1 | 2 |
| 3 | 0 | 1 |
| 4 | 2 | 2 |
| 5 | 1 | 2 |
| 6 | 2 | 2 |
| 7 | —* | 2 |
| 8 | 1 | 1 |
| 9 | 1 | 2 |
| 10 | 0 | 1 |
| 11 | 1 | 2 |
| 12 | 2 | 2 |

*No answer

All test persons observed a significant reduction in dandruff.

### Example 5

Composition B12 was evaluated for the treatment of Malassezia infected skin in the outer ears of an Irish soft coated wheaten terrier (dog age 11 years). Ears were gently washed with physiological salt water and the powder composition B12 was sprinkled into the ears and gentle massaged into the infected part of the outer ears 2 times per day. Significant effect of the treatment was observed already after 2 days with a reduction in blushing, moldy smell and skin irritation in the ear. After 7 days the treatment was reduced to once per day. After 2 weeks the infection in the outer ear was gone.

### Example 6

Effect of pH on the activity of the actives were determined using the spot on lawn method described in example 1. Lactiplantibacillus plantarum LB244R® (DSM 32996), Lactiplantibacillus plantarum LB356R® (DSM 33094) and Pediococcus pentosaceus LB606R® (DSM 33730) were each grown in MRS media at 30° C. for 48 hours, the supernatant from each culture were separated by centrifugation and filtered to create a cell free supernatant.

The supernatants were adjusted with 1M NaOH and 1M HCl respectively to the following pH values: 3, 3.5, 4, 4.5, 5, 5.5, 6, 6.5, 7, 7.5, 8. Phosphate buffer at each pH value was used as control for pH effect.

Proliferation of Malassezia was measured as inhibition zones using the spot on lawn method. All 3 supernatants were able to inhibit proliferation of Malassezia at pH below approximately 6.6.

### Example 7

Inhibition of M. furfur DSMZ 6170, M. restricta CBS 7877 and M. 41Lobose CBS 7874

Inhibition was assessed using a spot-on lawn method adapted from Zhang P. et al. (2015) Inter-strain interactions between bacteria isolated from vacuum-packaged refrigerated beef. Appl Environ Microbiol 81:2753-2761. doi:10.1128/AEM.03933-14 and Arena, M. P. et al. (2016) Use of Lactobacillus plantarum Strains as a Bio-Control Strategy against Food-Borne Pathogenic Microorganisms. Frontiers in Microbiology 7 (APR): 1-10. doi:10.3389/fmicb.2016.00464.

Target strain Malassezia furfur DSMZ 6170 was obtained from Leibniz Institute DSMZ. Target strains M. globosa CBS 7874 and M. restricta CBS 7877 were obtained from Westerdijk fungal biodiversity institute CBS. Target strains were grown in modified Leeming Notmann (mLN) (ATCC Medium No. 2737 Leeming & Notman agar Modified) agar and single colonies were inoculated into 10 ml of mLN Broth. A lawn was created using the target strain culture and allowed to dry. Bacterial strains isolated from example 1 were cultured from storage in 10 mL MRS broth. Subsequently, overnight cultures of LAB were spotted onto the plates and allowed to incubate. M. furfur was grown at 30° C. for approximately 10 days, M. restricta was grown at 30° C. for approximately 14 days and M. globosa was grown at 33° C. for approximate 14 days. Inhibition zones were measured from the edge of the LAB colony to the beginning of visible Malassezia growth (clearing zones) in millimeters. All plates were done in technical duplicates and repeated on different occasions.

LAB were screened against M. furfur DSMZ 6170, of which 84 showed moderate inhibition and 18 showed high inhibition. 2 strains were selected from the 18 strains and subsequently tested using the spot-on lawn method against M. restricta CBS 7877 and M. globosa CBS 7874. All 4 strains were capable of inhibiting M. restricta CBS 7877 and M. globosa CBS7874.

TABLE 1
Spot assay with Malassezia spp. As target, inhibition/clearing
zones measured in millimeters as an average
of duplicates between the periphery of the bacterial
colony and visible growth of
Malassezia spp..

| | Inhibition | Inhibition | Inhibition |
|---|---|---|---|
| | zone- | zone- | zone- |
| | M. furfur | M. restricta | M. globosa |
| | DSMZ | CBS | CBS |
| Strain | 6170 | 7877 | 7874 |
| L. paracasei LB555R | 3.0 | 20 | 21.5 |
| P. pentosaceus LB606R | 4.0 | 27 | 21 |
| L. plantarum LB681R | 4.5 | 30 | 17.5 |

### Example 8

Trichophyton Spp. Spot on Agar Assay

Anti-fungal activity against Trichophyton spp. were determined as described in example 7.

Trichophyton rubrum CBS 189.69 a nail isolate was obtained from the Westerdijk Fungal Biodiversity Institute part of the Royal Netherlands Academy of Arts and Sciences.

Trichophyton rubrum CBS 392.58 (Neotype of Epidermophyton rubrum Castell) a skin (foot) isolate was obtained from the Westerdijk Fungal Biodiversity Institute part of the Royal Netherlands Academy of Arts and Sciences.

Trichophyton spp. was grown on Sabouraud maltose agar at 24° C. for 2 to 3 weeks.

TABLE 2
Spot assay with Trichophyton spp. As target, inhibition/
clearing zones measured in millimeters as an average
of duplicates between the periphery of the bacterial colony
and visible growth of Trichophyton spp

| | Inhibition zone- | Inhibition zone- |
|---|---|---|
| Strain | T. rubrum CBS 189.69 | T. rubrum CBS 392.58 |
| L. paracasei LB555R | 28 | 23 |
| L. plantarum LB681R | 35 | 25 |
| P. pentosaceus LB606R | 24 | 20 |

### Example 9

### Inhibition by Cell-Free Supernatant Solution

Growth of Malassezia spp. (3 strains as described in example 7) with and without cell free supernatant (CFS) of LAB from example 2 was monitored using the oCelloScope (BioSense Solutions ApS, Denmark).

Between 5 to 10 mL of LAB cell culture was centrifuged (2,700×g, 10 min) and sterile filtered using a 0.2 μm syringe filter. Samples were taken from the LAB-cultures after 24-72 hours. From 10 mL of Malassezia spp. Cultures a 1:2 dilution was prepared. From each of these dilutions, 100 μL was mixed with 100 μL CFS or dilutions thereof using of 100, 75, 50, 25, 10 and 0% CFS in a 96 wells microtiter plate. The volume was adjusted using MRS broth. As a positive control for inhibition, Malassezia spp. Were treated with fluconazole (64 μg/mL). All challenge assays were run in technical triplicates and repeated on separate occasions. The plate was incubated in the oCelloScope at 37° C. Raw images were taken every 2 and a half hours for up to 48 hours and growth curves were plotted in excel, calculated as an average of the triplicates.

Post-experiment, plates were visually inspected, and raw images were visually examined. Images were analysed using UniExplorer PC software.

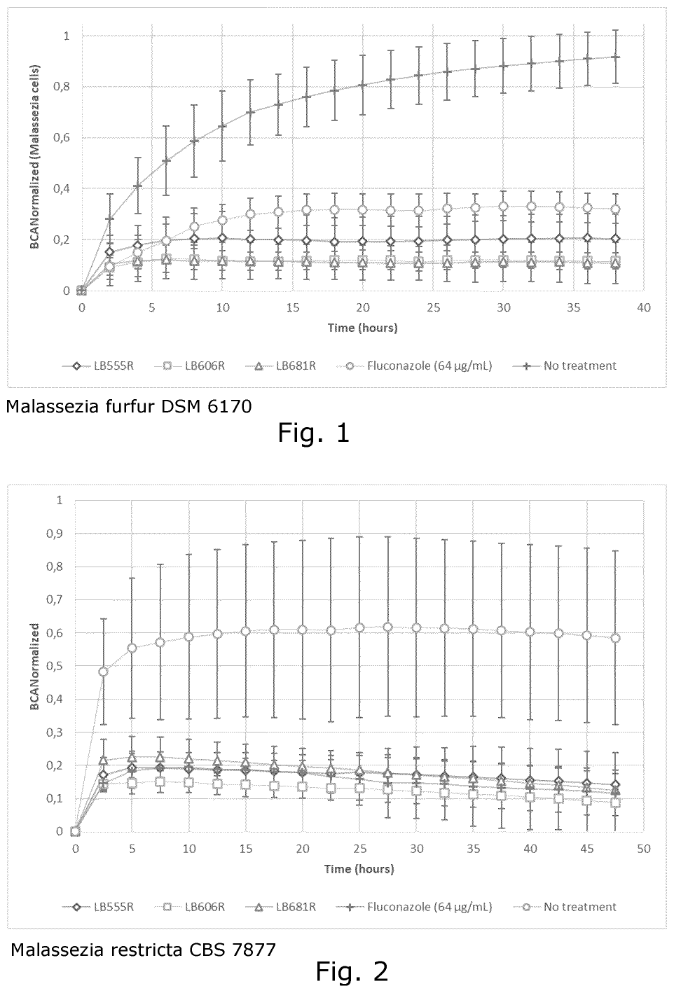

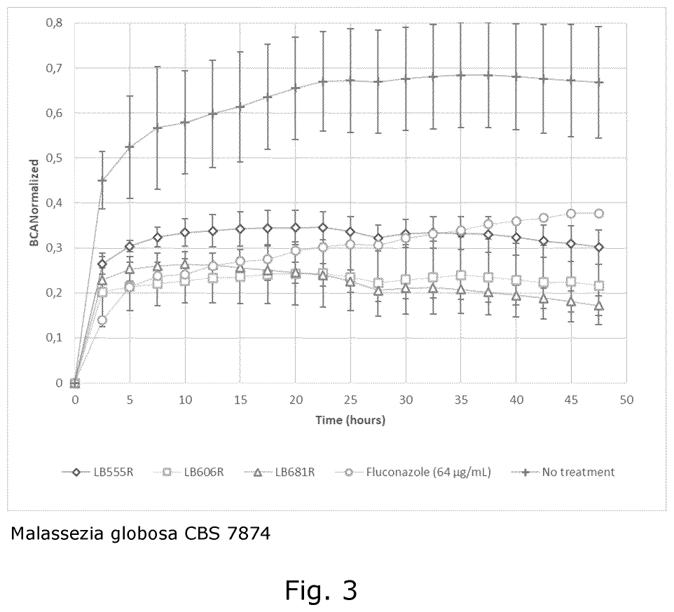

The results are shown in FIGS. 1-3 and demonstrated that the cell free supernatants obtained from three isolated strains (LB555R, LB681R, and LB606R) have a strong and significant inhibition of Malassezia furfur DSM 6170 (FIG. 1), M. restricta CBS 7877 (FIG. 2) and M. globosa CBS7874 (FIG. 3) using a composition comprising: (i) Lacticaseibacillus paracasei subsp. paracasei LB555R, deposited under the assession number DSM 34249; (ii) Lactiplantibacillus plantarum, LB681R, deposited under the assession number DSM 34250; (iii) Pediococcus pentosaceus LB606R (DSM 33730), compared to a composition comprising the anti-fungal compound Fluconazole (in a concentration of 64 μg/ml); and compared to an un-treated fungal solution.

3 hit strains were identified in the screening as the lactic acid bacteria being able to growth inhibit the fungal growth of at least 2 pathogenic fungi, wherein the growth inhibition was significantly better than what was observed for the known antifungal antibiotic Fluconazole.

### Example 10

### Analysis of Short Chain Fatty Acid Production (SCFA)

Sample analysis was carried out as follows. Cell free supernatant from four strains (LB555R; LB681R; LB606R; and LB244R) grown in MRS medium 24 hours at 37° C. were acidified using hydrochloride acid, and deuterium labelled internal standards where added. Analysis was performed using a high polarity column (Zebron™ ZB-FFAP, GC Cap. Column 30 m×0.25 mm×0.25 μm) installed in a GC (7890B, Agilent) coupled with a quadropole detector (5977B, Agilent). The system was controlled by ChemStation (Agilent). Raw data was converted to netCDF format using Chemstation (Agilent), before the data was imported and processed in Matlab R2014b (Mathworks, Inc.) using the PARADISe software described by Johnsen et. al (DOI: 10.1016/j.chroma.2017.04.052). The SCFA method is a GC-MS method specially targeted to short-chain fatty acids using a high polarity column and standards. Concentration of short chain fatty acids (acetic acid) was determined in the supernatant from each strain

| | LB555R | LB681R | LB606R | LB244R |
|---|---|---|---|---|
| acid | 36.5 mM | 40.76 mM | 45.8 mM | 48.0 mM |

### Example 11

### Different Topical Formulations for Skin and Nails

Loose powder, leave-on formulation

### Ingredients:

Oryza sative (rice) starch 49% (w/w)
Tapioca starch 49% (w/w)
Lacticaseibacillus paracasei subsp. paracasei LB555R 2% (w/w) (Lyophilized powder with the concentration of 9×10⁹ CFU/g)

### Loose Powder, Leave-on Formulation

### Ingredients:

Zea mays (corn) starch 49% (w/w)
Tapioca starch 49% (w/w)
Lactiplantibacillus plantarum LB830R 2% (w/w) (Lyophilized powder with the concentration of 8×10⁹ CFU/g)

### Topical Liquid Ointment

### Ingredients:

Water 90% (w/w)
Ferment lysate/Postbiotic (Lactiplantibacillus plantarum, LB681R) 10% (w/w)

### Topical Oil Formulation:

### Ingredients:

Jojoba oil 48% (w/w)
Sunflower oil 48% (w/w)
Lactiplantibacillus plantarum, LB681R 1% (w/w) (Lyophilized powder with the concentration of 5×10⁹ CFU/g)
Lacticaseibacillus paracasei subsp. paracasei LB555R 1% (w/w) (Lyophilized powder with the concentration of 9×10⁹ CFU/g)
Inulin (FOS) 1% (w/w)
Tocopherol 1% (w/w)

### Example 12

### Well Diffusion Assay

Overnight cultures of LAB were prepared. LAB was grown in MRS broth and the fungi tested grown as described in the examples above for spot on lawn. Wells were made in the plates and 50 UL of the LAB cultures were transferred to the wells. After incubation until visible fungal growth, inhibition zones around the wells were examined.

TABLE 3
Spot on lawn and diffusion assay, inhibition
zones are measured in mm.

| | Spot on lawn | | Well diffusion | |
|---|---|---|---|---|
| | (Diameter in mm) | | (Radius mm) | |
| | LB555R | LB681R | LB555R | LB681R |
| T. rubrum CBS 189.69 | 25 +/− 5 | 30 +/− 8 | 6 +/− 1 | 7 +/− 1 |
| T. rubrum CBS 392.58 | 12 +/− 4 | 18 +/− 4 | 7 +/− 2 | 6 +/− 2 |
| M. furfur DSMZ 6170 | 3 +/− 1 | 4.5 +/− 2 | 5 +/− 1 | 5 +/− 1 |

### REFERENCES

U.S. Pat. No. 4,654,207
U.S. Pat. No. 5,019,376
U.S. Pat. No. 3,929,678
U.S. Pat. No. 2,658,072
U.S. Pat. No. 2,438,091
U.S. Pat. No. 2,528,378
U.S. Pat. No. 5,104,646
U.S. Pat. No. 5,106,609
WO 2011/138450
FR 2908045
U.S. Pat. No. 4,565,647
The CTFA Cosmetic Ingredient Handbook (2004)

1. A composition comprising:
one or more isolated probiotic bacterial strains; two or more active of the one or more isolated probiotic bacterial strains; or the combination of one or more isolated probiotic bacterial strains and two or more active of the one or more isolated probiotic bacterial strains, and
an acceptable carrier,
wherein the one or more isolated probiotic bacterial strains and/or the two or more active of the one or more isolated probiotic bacterial strains is capable of inhibiting fungal proliferation, wherein the composition comprises a pH below 6.5.

2. The composition according to claim 1, wherein the composition comprises nucleic acid and/or nucleotides.

3. The composition according to claim 1, wherein the composition comprises no plant material and/or fibre material.

4. The composition according to anyone of claims 1-3, wherein the composition has a pH value below pH 5.5.

5. The composition according to claim 1, wherein the at least two actives are produced by metabolism of the isolated viable probiotic bacterial strain.

6. The composition according to claim 1, wherein the least two actives are produced by a single isolated probiotic bacterial strain.

7. The composition according to claim 1, wherein the actives are selected from a bacteriocins, an organic acid, a cell wall material, or a combination hereof.

8. A composition according to claim 1, for the prevention and/or treatment of mycoses and/or dandruff in a human or in an animal.

9. The composition according to claim 8, wherein the prevention and/or treatment of mycoses and/or dandruff in a human or in an animal is prevention and/or treatment of conditions associated with the proliferation of yeasts of the Malassezia genus on a scalp, on hands, on ears, on feet, on nails or on skin of a subject.

10. A topical hair care composition comprising a combination of the composition as claimed in claim 1 and a shampoo matrix with pH below 6.5 comprising at least one detersive surfactant selected from the group consisting of an anionic surfactant, a nonionic surfactant, an amphoteric surfactant, or a combination thereof.

11. A composition comprising:
one or more isolated viable probiotic bacterial strains, and
an acceptable carrier,
wherein the one or more isolated probiotic bacterial strains is capable of inhibiting fungal proliferation, wherein the composition is formulated into a powder.

12. The composition according to claim 11, for treatment of dandruff and/or mycosis wherein the composition comprises viable probiotic microorganisms in at least a concentration of 10³ colony forming unit per gram of composition wherein the probiotic microorganism is able to produce anti-dandruff and/or anti-mycosis actives

13. The composition according to claim 11, wherein the composition is a dry-shampoo.

14. The composition according to claim 11, wherein for scalp treatment, skin treatment and diaper rash.

15. The composition according to claim 1, wherein the composition comprising viable probiotic bacteria in a concentration from 10³ to 10¹³ colony forming units of bacteria per gram.

16. The composition according to claim 1, wherein the composition comprises a pH in the range of 3.5-5.5.
